(19) United States
(12) Patent Application Publication
(10) Pub. No.: US 2024/0423655 A1
(43) Pub. Date: Dec. 26, 2024

(54) CONTROL OF IVL SYSTEMS, METHODS AND DEVICES

(71) Applicant: Nextern Innovation, LLC, White Bear Township, MN (US)

(72) Inventors: Austin P. Petronack, Plymouth, MN (US); Jason W. Staab, Apple Valley, MN (US); John R. Ballard, Waconia, MN (US); J. Samuel Batchelder, Woodinville, WA (US); Jacob T. Williams, Plymouth, MN (US); Donald D. Hanson, Chaska, MN (US); Thomas D. Brindley, Plynouth, MN (US); Scott P. Boeshart, Plymouth, MN (US); Jeffrey R. Moriarty, Roseville, MN (US)

(21) Appl. No.: 18/825,683

(22) Filed: Sep. 5, 2024

Related U.S. Application Data
(63) Continuation-in-part of application No. 18/506,305, filed on Nov. 10, 2023.
(60) Provisional application No. 63/424,573, filed on Nov. 11, 2022.
(60) Provisional application No. 63/650,872, filed on May 22, 2024.
(60) Provisional application No. 63/580,547, filed on Sep. 5, 2023.

(51) Int. Cl. A61B 17/22

(52) U.S. Cl. CPC A61B 17/22022

(57) ABSTRACT

Various embodiments of the systems, methods, and devices are provided for controlled operation of IVL for breaking up calcified lesions in an anatomical conduit. More specifically, control arrangements are disclosed concerning managing and/or providing electrical energy to generate an electrical arc between a set of spaced-apart electrodes disposed within a fluid-filled balloon, creating stable and constant, or slightly increasing, pressure output over at least 300 voltage pulses. Control arrangements disclosed further determine whether an electrical arc was successfully generated.

### CROSS-REFERENCE TO RELATED APPLICATIONS

This application is a continuation-in-part of U.S. utility application Ser. No. 18/506,305, filed Nov. 10, 2023, entitled CONTROL OF IVL SYSTEMS, DEVICES AND METHODS THEREOF which claims the benefit of provisional application No. 63/424,573 filed Nov. 11, 2022, this application also claims the benefit of provisional application No. 63/650,872, filed May 22, 2024 entitled CONTROL OF IVL SYSTEMS, METHODS AND DEVICES, provisional application No. 63/580,547, filed Sep. 5, 2023 entitled DEVICES, SYSTEMS, AND METHODS OF INTRAVASCULAR LITHOTRIPSY, PCT application number PCT/US2023/79209 filed Nov. 9, 2023 entitled CONTROL OF IVL SYSTEMS, DEVICES AND METHODS AND DEVICES, and PCT/US2023/085868, filed Dec. 23, 2023 entitled INTRAVASCULAR LITHOTRIPSY SYSTEM WITH IMPROVED DURABILITY, EFFICIENCY AND PRESSURE OUTPUT VARIABILITY, the entire contents of each of which are incorporated herein by reference.

### STATEMENT REGARDING FEDERALLY SPONSORED RESEARCH OR DEVELOPMENT

None

### BACKGROUND OF THE INVENTION

### Field of the Invention

The present disclosure relates to devices, systems, and methods for breaking up calcified lesions in an anatomical conduit. More specifically, the present disclosure relates to devices, systems, and methods for applying electrical arc spaced-apart electrodes disposed within a fluid-filled member to creating flow and pressure waves.

### Description of the Related Art

A variety of techniques and instruments have been developed for use in the removal or repair of tissue in arteries and similar body passageways, including removal and/or cracking of calcified lesions within the passageway and/or formed within the wall defining the passageway. A frequent objective of such techniques and instruments is the removal of atherosclerotic plaque in a patient's arteries. Atherosclerosis is characterized by the buildup of fatty deposits (atheromas) in the intimal layer (i.e., under the endothelium) of a patient's blood vessels. Very often over time what initially is deposited as relatively soft, cholesterol-rich atheromatous material hardens into a calcified atherosclerotic plaque, often within the vessel wall. Such atheromas restrict the flow of blood, cause the vessel to be less compliant than normal, and therefore often are referred to as stenotic lesions or stenoses, the blocking material being referred to as stenotic material. If left untreated, such stenoses can cause angina, hypertension, myocardial infarction, strokes and the like.

Angioplasty, or balloon angioplasty, is an endovascular procedure to treat by widening narrowed or obstructed arteries or veins, typically to treat arterial atherosclerosis. A collapsed balloon is typically passed through a pre-positioned catheter and over a guide wire into the narrowed occlusion and then inflated to a fixed pressure. The balloon forces expansion of the occlusion within the vessel and the surrounding muscular wall until the occlusion yields from the radial force applied by the expanding balloon, opening up the blood vessel with a lumen inner diameter that is similar to the native vessel in the occlusion area and, thereby, improving blood flow.

The angioplasty procedure presents some risks and complications, including but not limited to: arterial rupture or other damage to the vessel wall tissue from over-inflation of the balloon catheter, the use of an inappropriately large or stiff balloon, the presence of a calcified target vessel; and/or hematoma or pseudoaneurysm formation at the access site. Generally, the pressures produced by traditional balloon angioplasty systems is in the range of 10-15 atm, but pressures may at times be higher. As described above, the primary problem with known angioplasty systems and methods is that the occlusion yields over a relatively short time period at high stress and strain rate, often resulting in damage or dissection of the conduit, e.g., blood vessel, wall tissue.

Traditional systems may apply coarse system controls. For example, ceasing power supply from the power source may be applied as a primary means to regulate the amount of energy applied to a therapy site. Such an approach is taught by U.S. Pat. No. 8,728,091 wherein current is monitored during application of voltage by a pulse generator. When the current exceeds a predetermined threshold magnitude, the voltage is turned off at the pulse generator. As discussed in additional detail herein, a refined approach to power characteristic control can assist in more effective and more consistent therapy. For example, improved durability, higher frequency and substantially equivalent pressure output, over a longer number of voltage pulses than previously possible, are provided using embodiments of the present disclosure.

Various embodiments of the present disclosure can address these issues, among others, discussed above.

### BRIEF DESCRIPTION OF THE SEVERAL VIEWS OF THE DRAWINGS

These drawings are exemplary illustrations of certain embodiments and, as such, are not intended to limit the disclosure.

FIG. 1 illustrates a schematic intravascular lithotripsy (IVL) arrangement according to one or more embodiments of the present disclosure.

FIG. 2 illustrates a flowchart for controlling and delivering voltage to electrodes and generating pressure waves according to one or more embodiments of the present disclosure.

FIG. 3A illustrates portions of circuitry arrangements of one or more of the IVL arrangement and control operation of FIGS. 1 and 2 according to one or more embodiments of the present disclosure.

FIG. 3B illustrates portions of circuitry arrangements of one or more of the IVL arrangement and control operation of FIGS. 1 and 2 according to one or more embodiments of the present disclosure.

FIG. 4A illustrates additional portions of circuitry arrangements of one or more of the IVL arrangement and control operation of FIGS. 1 and 2 according to one or more embodiments of the present disclosure.

FIG. 4B illustrates additional portions of circuitry arrangements of one or more of the IVL arrangement and control operation of FIGS. 1 and 2 according to one or more embodiments of the present disclosure.

FIG. 4C illustrates additional portions of circuitry arrangements of one or more of the IVL arrangement and control operation of FIGS. 1 and 2 according to one or more embodiments of the present disclosure.

FIG. 4D illustrates additional portions of circuitry arrangements of one or more of the IVL arrangement and control operation of FIGS. 1 and 2 according to one or more embodiments of the present disclosure.

FIG. 5 illustrates a flowchart for controlling and delivering voltage to electrodes and generating shock waves according to one or more embodiments of the present disclosure.

FIG. 6 illustrates a control operation flow according to one or more embodiments of the present disclosure

FIG. 7 illustrates graphic comparisons of voltages applied to a known IVL device and to embodiments of IVL devices according to the present disclosure.

FIG. 8 illustrates a graphic comparison of average peak pressure generated over 80 voltage pulses by a known IVL devices and embodiments of IVL devices according to the present disclosure.

FIG. 9 illustrates a graphic comparison of average peak pressure generated over 80 voltage pulses by a known IVL device and over a predetermined maximum number of voltage pulses by embodiments of IVL devices according to the present disclosure.

FIG. 10 illustrates an exemplary flowchart method according to the present disclosure.

FIG. 11 illustrates an exemplary flowchart according to the present disclosure.

### DETAILED DESCRIPTION OF THE INVENTION

Traditional intravascular lithotripsy (IVL) devices, systems, and methods can apply high energy, electrical power to generate a spark (arc) between discharge electrodes. Under appropriate conditions, sparks generated by submerged electrodes can generate pressure waves within the medium which can be applied to treat (breakup) calcified lesions within the patient's vasculature. It can be appreciated that appropriate control of such high energy systems can be paramount to provision of effective and safe treatment.

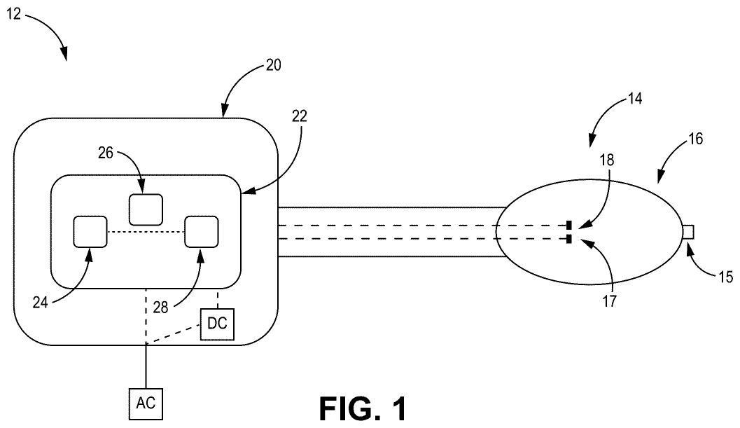

Referring to FIG. 1, a diagrammatic layout of portions of an exemplary IVL system 12 is shown indicating control elements within the present disclosure. The illustrative IVL system 12 comprises a catheter assembly 14 including an elongate body, embodied as a catheter having guidewire 15, and a fluid-fillable member, e.g., an exemplary and illustrative inflatable balloon 16 disposed near one end of the body and arranged to receive fluid for inflation to facilitate IVL therapy. A set of spaced-apart electrodes 18 are shown arranged within the balloon 16, at least some of which are spaced apart by a gap 17 from each other to create an electrical arc across the gap 17 between the spaced-apart electrodes 18.

The spaced-apart electrodes 18 are arranged in communication (as suggested by dashed line conductors) with an electric pulse generation system 20 to receive high voltage electrical energy for spark generation to create pressure waves for IVL therapy. In the illustrative embodiment, one electrode may be grounded and the other provided with high voltage from the electric pulse generation system 20, although in some embodiments, any voltage differential may be applied. The electric pulse generation system 20 includes an IVL control system 22 that may comprise a processor 24, executing instructions stored on memory 26 and communications signals via circuitry 28 for IVL operations according to the processor governance. The processor 24, memory 26, and circuitry 28 are arranged in communication with each other (as suggested via dashed lines) to facilitate disclosed operations.

Appropriate control of such high energy systems can also require achieving sufficient energy at the spaced-apart electrodes. Given the high energy environment and microscale time periods for electronic discharge, desirable energy control within such IVL devices and systems can be challenging. Moreover, adaptable control methodologies may offer advantages to IVL effectiveness. Adjustable energy delivery can increase efficient power application, which can reduce risk to the patient. For example, beginning with a predetermined starting voltage threshold and defining a predetermined upper voltage threshold to form an acceptable voltage window may be proved. The acceptable voltage window may be coupled with one or more series of two or more generated voltage pulses of magnitudes that are confirmed to be within the acceptable voltage window. If, e.g., the magnitudes of the series of generated voltage pulses are below the predetermined upper voltage threshold, then the target voltage may be increased by a predetermined amount and another series of generated voltage pulses is executed. In other embodiments, the duration of voltage application may be modified, e.g., increased, by a predetermined amount either alone or in combination with an increased target voltage.

Various embodiments may comprise IVF control systems and methods described in PCT application number PCT/US2023/79209 filed Nov. 9, 2023 entitled CONTROL OF IVL SYSTEMS, DEVICES AND METHODS AND DEVICES, U.S. utility application Ser. No. 18/506,305, filed Nov. 10, 2023, entitled CONTROL OF IVL SYSTEMS, DEVICES AND METHODS THEREOF, and PCT/US2023/085868, filed Dec. 23, 2023 entitled INTRAVASCULAR LITHOTRIPSY SYSTEM WITH IMPROVED DURABILITY, EFFICIENCY AND PRESSURE OUTPUT VARIABILITY, the entire contents of which is hereby incorporated by reference.

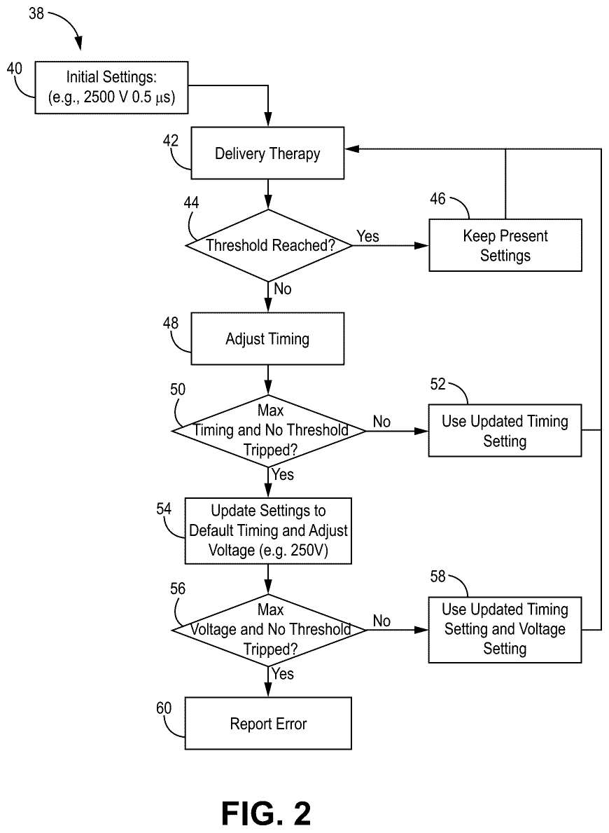

Referring now to FIG. 2, an exemplary flow diagram is shown concerning control 100 in operation of an IVL system, specifically the number of voltage pulses generated and the magnitude of the generated pulses. Such control operations can be governed by the electric pulse generation system 20, and illustratively by the IVL control system 22 discussed above in connection with FIG. 1.

Referring now to FIG. 2, a flow diagram is shown concerning one embodiment of a control 38 in operation of IVL as discussed concerning boxes 40 through 60. Such control operations can be governed by the electric pulse generation system 20, and illustratively by the IVL control system 22. As discussed in additional detail herein, control 38 applies incremental changes in power parameters during cycling of applied voltage while monitoring parameters related to spark generation. For example, incrementally increasing the magnitude of the voltage that is applied to the discharge electrodes, and/or the duration of the voltage application, can increase the likelihood of generation of an effective spark without excessive energy.

Moreover, if increases to the duration of voltage applied to the discharge electrodes fails to generate sufficient spark, incremental increase to the voltage can further increase the likelihood of generation of an effective spark without excessive power. Still further, if incrementally increased voltage fails to generate sufficient spark, duration can once again be incrementally increased before further increasing voltage. Accordingly, it can be appreciated that controlled incremental increases in duration and voltage can be implemented to achieve effective spark generation, and thus pressure wave generation, at or near the lowest required power characteristics for effective spark generation. Increasing the likelihood of reaching sufficient spark with lower power can increase the efficiency, safety, and/or reduce intensity of effective IVL therapy.

In box 40, initial settings comprising an initial target voltage magnitude and duration of application are applied. For illustrative example, default initial settings are applied as a discharge voltage of 2500 volts (V) for a duration of 0.5 microseconds. In some embodiments, the initial settings may be determined by any suitable manner, including by use of programmable defaults, as adjusted settings based on use, for example, adjusted based on the patient characteristics, environmental conditions, procedural approach, and/or product cycle lifetime (i.e., operable age), among other aspects. From box 40, control may proceed to box 42.

In box 42, electrical energy is applied to the spaced-apart electrodes to deliver IVL therapy. In the first instance of proceeding from box 40 to box 42, electrical energy is applied at the initial settings, e.g., an applied voltage of 2500 V for a duration of 0.5 microseconds. When applied in a clinical setting, such that the IVL catheter is arranged within the patient's body lumen, and specifically with discharge electrodes submerged within a fluid medium within a fluid-filled member such as an angioplasty balloon, pressure wave therapy can ensue. However, as mentioned below, at present conditions, in some instances the energy provided at the initial settings may be insufficient to generate a spark at the electrodes, or may generate insufficient spark or insufficient pressure wave. From box 42, control may proceed to box 44.

In box 44, determination of threshold aspects is conducted. In one embodiment, a determined value for current applied in box 42 is compared with a predetermined threshold current value to determine and ensure that an electrical arc was formed between the spaced-apart electrodes. The predetermined threshold current value may be embodied as a predetermined fixed value, e.g., 20 amperes (amps), but in some embodiments, may have any suitable value, for example 50 amps, 100 amps, 150 amps, 175 amps, the threshold current value for a given cycle may be determined based on the number of previous cycles in a therapy session, the number of cycles maintaining present settings (e.g., through box 46) in a therapy session, the number of particular successive cycles (e.g., number of successive cycles through box 46 or box 48 or box 52 or box 54 or box 58), the patient characteristics, environmental conditions, procedural approach, and/or product cycle lifetime (i.e., operable age), among other aspects. Additionally, although the threshold current value may be set to one value, the actual current passed during a sufficient generation of arc across the electrodes may be greater. The mechanism for monitoring the current applied is discussed further below.

Responsive to determination that the determined value for current applied in box 42 is equal to or greater than the threshold current value, control may proceed to box 46. Otherwise, responsive to determination that the determined value for current applied in box 42 less than the threshold current value, control may proceed to box 48. For example, as mentioned above, insufficient spark generation may result in little or no current flow (e.g., about 0 to about 5 amps, which may represent dissipated energy into the medium without arcing) across the electrodes, which would not achieve the threshold current value, and thus would proceed to box 48.

In the illustrative embodiment, the determined current value for current applied in box 42 is embodied as the instantaneous current applied across the electrodes and may be determined for each applied voltage pulse. In some embodiments, the determined current value may be embodied as an aggregated value, such as a time-averaged value of current applied to the electrodes. Continuing from the exemplary embodiment applying a threshold current value of 20 amps, when 20 amps is achieved by the therapy delivered, the electrical arc is determined to have occurred and the initial settings of voltage magnitude and duration are sufficient to produce the desired electrical arc.

It can be appreciated that the substantial occurrence of 20 amps provides considerable current across the discharge electrodes indicating the generation of meaningful spark. By comparison little or no current may flow across the discharge electrode when a spark fails to generate or when insufficient spark is generated under the applied duration and at the applicable voltage, 2500 V by example, and which can generally range between about 500 V to about 5000 V for IVL therapy, although conceivably can range between about 100 V to about 10000 V in terms of practical application.

In box 46, determination is made to maintain presently selected settings. In the illustrative embodiment, the presently selected settings include the discharge voltage of and applicable duration as applied immediately previously in box 42. For avoidance of doubt, if the initial settings have just immediately been applied in box 42, resulting in 20 amps of current, the initial settings would be applied to the next voltage pulse and/or cycle or series of voltage pulses. However, if the presently selected settings include updated settings, for example, updated duration and/or voltage settings from later portions of control 38, as discussed in additional detail herein, then maintaining presently selected settings would include the immediately applied updated settings. Maintaining presently selected settings proceeds openly to return to box 42, to again apply electrical energy to the electrodes to deliver IVL therapy.

In box 48, if the current threshold is not met then a determination is made to increase the duration of applied voltage to the discharge electrodes. Continuing the example in which less than 20 amps of current indicates insufficient spark generation, rather than immediately increasing the voltage applied, the duration of applied voltage can be incrementally increased. At the microsecond-scale, increasing the duration of applied voltage can increase the likelihood of sufficient spark generation using the same voltage previously applied. This can be achieved as the result of overcoming threshold system impendence and/or other factors affecting the ease of spark generation during a given cycle at a given voltage.

The duration of applied voltage may be increased by a predetermined duration interval, illustratively embodied as a fixed value, e.g., 0.5 microseconds or other duration increase. In some embodiments, the predetermined interval for a given voltage pulse and/or cycle or series of voltage pulses may be determined based on factors such as the number of times therapy cycles have ensued previously during the therapy session (e.g., number of voltage pulses, cycles or series intervals have proceeding through boxes 42, 44, and 46, prior to proceeding to box 48), the patient characteristics, environmental conditions, procedural approach, and/or product cycle lifetime (i.e., operable age), among other aspects. In some embodiments, the predetermined duration interval for a given cycle may be varied by predetermined rate of change, for example, by percentage gain or loss by cycle. Control 38 then proceeds openly from box 48 to box 50.

In box 50, determination is made whether a maximum duration has been achieved. In some embodiments, the maximum duration is a predetermined duration embodied as a fixed value, e.g., 35 or more microseconds. By way of example, if the control sequence progresses through cycles from initial settings of box 40 at 0.5 microseconds through box 48, until reaching an exemplary maximum of 35 microseconds, the maximum duration would be achieved as a threshold value.

In some embodiments, the maximum duration for a given cycle may be determined based on the number of previous cycles in a therapy session, the number of cycles maintaining present settings (e.g., through box 46) in a therapy session, the number of particular successive cycles (e.g., number of successive cycles through box 46 or box 48 or box 52 or box 54 or box 58), the patient characteristics, environmental conditions, procedural approach, and/or product cycle lifetime (i.e., operable age), among other aspects. By deduction, in box 50, the threshold current value has not been achieved in the present cycle, although in some embodiments, affirmative determination and/or confirmation that threshold current value has not been achieved may be performed. Responsive to determination that the maximum duration has not been achieved, control proceeds to box 52. Otherwise, responsive to determination that maximum duration has been achieved, control proceeds to box 54.

In box 52, determination is made to apply the updated duration and return to delivery of therapy in box 42. In the illustrative embodiment, the duration has been updated in box 48 by increasing the presently selected duration by the predetermined duration interval, and determination to apply the updated duration confirms and proceeds with the updated duration. In the illustrative embodiment, the applied voltage remains as presently selected. Proceeding with the updated duration proceeds openly to return to box 42, to again apply electrical energy to the discharge electrodes to delivery therapy using the updated duration.

In box 54, the applied voltage is increased by a predetermined voltage interval. The presently selected setting for applied voltage is illustratively increased by the predetermined voltage interval. The presently selected duration is returned to the value at the initial setting, exampled as 0.5 microseconds, to be applied with the updated voltage, although in some embodiments, the updated duration may have any suitable value under newly updated voltage settings, for example, the updated duration may be determined based on the number of cycles of the therapy session when newly update voltages occur.

In the illustrative embodiment, the predetermined voltage interval is embodied as a fixed value of 250 V, such that, in the first exemplary occurrence of box 54, the presently selected applied voltage is increased to 2750 V from the value at the initial setting of 2500 V. In some embodiments, the predetermined voltage interval for a given cycle may be determined based on the number of previous cycles in a therapy session, the number of cycles maintaining present settings (e.g., through box 46) in a therapy session, the number of particular successive cycles (e.g., number of successive cycles through box 46 or box 48 or box 52 or box 54 or box 58), the patient characteristics, environmental conditions, procedural approach, and/or product cycle lifetime (i.e., operable age), among other aspects.

In box 56, determination is made whether maximum voltage has been achieved. In the illustrative embodiment, the maximum voltage is a predetermined voltage embodied as a fixed value, e.g., 3500 V. By way of example, if the control sequence progressed through cycles from initial settings of box 40 at 2500 V through box 54, until reaching 3500 V, the maximum voltage would be achieved as a threshold value.

In some embodiments, the maximum voltage for a given cycle may be determined based on the number of previous cycles in a therapy session, the number of cycles maintaining present settings (e.g., through box 46) in a therapy session, the number of particular successive cycles (e.g., number of successive cycles through box 46 or box 48 or box 52 or box 54 or box 58), the patient characteristics, environmental conditions, procedural approach, and/or product cycle lifetime (i.e., operable age), among other aspects. By deduction, in box 56, the threshold current value has not been achieved in the present cycle, although in some embodiments, affirmative determination and/or confirmation that threshold current value has not been achieved may be performed. Responsive to determination that maximum voltage has not been achieved, control proceeds to box 58. Otherwise, responsive to determination that maximum voltage has been achieved, control proceeds to box 60.

In box 58, determination is made to apply updated voltage. In the illustrative embodiment, the voltage has been updated in box 54 by increasing the presently selected voltage by the predetermined voltage interval, and determination to apply the updated voltage confirms and proceeds with the updated voltage. The duration was updated in box 45 to return to the value at the initial setting, exampled as 0.5 microseconds, and proceeds to be applied with the updated applied voltage. Proceeding with the updated voltage proceeds openly to return to box 42, to again apply electrical power to the electrodes to delivery therapy using the updated voltage and updated duration.

In box 60, determination of error occurs. Responsive to determination of error, an error message is provided. Such an error message illustratively terminates the therapy session, but in some embodiments, may take other safety and/or communication actions, for example, such as displaying an error communication to a user for consideration in determining remedial action.

In the illustrative embodiment, by deduction, responsive to error determination, the maximum voltage and maximum duration can be determined not to have generated a spark, although in some embodiments, insufficient spark generation may be determined. In some embodiments, responsive to error determination, failed and/or insufficient spark generation under maximum voltage and maximum duration may be determined by affirmative determination and/or confirmation. Following box 60, process control automatically terminate.

Within the discussion of the control 38, exemplary increases in duration have been mentioned, although in some instances, for example, in certain cycles of control 38, voltage level may be decreased, for example, in certain cycles of control 38. For example, duration and/or voltage may be changed in a given cycle according to control 38 to decrease incrementally to achieve appropriate conditions, for example, to achieve appropriate discharged energy as discussed in additional detail herein relative to consideration of the voltage levels of an energy storage system, for example, a capacitance system before and after discharge.

As discussed further herein, control system 22 may control the number of generated voltage pulses in a series (or a plurality of series) of voltage pulses. An acceptable voltage window comprises a predetermined starting voltage magnitude and a predetermined upper voltage magnitude. IVL control system further comprises a predetermined voltage magnitude for incremental increasing of voltage magnitude after each series of voltage pulses if the magnitudes of the executed voltage pulses is within the acceptable voltage window. Separate sets of predetermined control data may be provided in control system 22 for IVL systems comprising balloons that are of identifiable characteristics such as, without limitation, different outer diameter sizing, e.g, 2.5 mm, 3.0 mm, 3.5 mm and/or 4.0 mm.

An exemplary embodiment may comprise a 2.5 mm or a 3.0 mm balloon, wherein the control system 22 comprises control data including an exemplary starting target voltage of about 3000V (predetermined lower voltage threshold), voltage pulse series comprising 10 pulses, and an incremental voltage increase of 25V if the voltage pulse series magnitudes are less than an exemplary upper voltage threshold of about 3500V. As the skilled artisan will recognize, the incremental voltage increase may comprise any voltage magnitude, including without limitation within about 1V to about 250V. A preferred voltage increase may comprise about 25V, but may be greater or less than 25V in certain embodiments. As the artisan will recognize, the predetermined starting voltage may be less than 3000V and the predetermined upper voltage threshold may be greater than 3500V. Thus, an exemplary starting voltage may, without limitation, comprise about 2500V and an exemplary upper voltage threshold may comprise about 4100V. In other embodiments, an exemplary predetermined starting voltage maybe greater than 3000V and an exemplary upper voltage threshold may be greater than about 3250V.

Another exemplary embodiment may comprise a 3.5 mm or a 4.0 mm balloon, wherein the control system 22 comprises control data including an exemplary starting target voltage of 2850V (predetermined lower voltage threshold), voltage pulse series comprising 10 pulses, and an incremental voltage increase of 25V if the voltage pulse series magnitudes are less than an exemplary predetermined upper voltage threshold of 3700V.

Another exemplary embodiment may comprise a 2.5 mm, 3.0 mm, 3.5 mm or 4.0 mm wherein the control system 22 comprises control data including an exemplary predetermined starting target voltage of about 2850V and an exemplary upper voltage threshold of about 3250V.

In some embodiments, the control system 22 may comprise an exemplary predetermined starting voltage magnitude and an exemplary upper voltage threshold that are within the range of about 2700V to about 3700V.

In some embodiments, the current produced by a voltage pulse maybe within the range of 120 amps to 220 amps.

In some embodiments, a series of voltage pulses may be generated for delivery to one or more sets of spaced-apart electrodes 18, followed by a pause in generation of voltage delivery. Certain embodiments may comprise a pause between a last pulse in a series of voltage pulses and a first pulse in an immediately subsequent series of voltage pulses that is within the range of 5-20 seconds. More preferably, the pause between adjacent voltage pulse series may be about 10 seconds.

Some embodiments may comprise a predetermined time gap or duration between adjacent voltage pulses within a series of voltage pulses. In some embodiments, the time gap or duration between adjacent voltage pulses within a series of voltage pulses may be within the range of about 0.25 seconds (correspondent to a voltage pulse frequency of about 4 Hz) to about 1 second (correspondent to a voltage pulse frequency of about 1 Hz).

In some embodiments, a preferred time gap or duration between adjacent voltage pulses within a series of voltage pulses may be about 0.5 seconds (corresponded to about 2 Hz).

In some embodiments, the pause of predetermined duration between adjacent series of voltage pulses may be greater in length or duration than the time gap or duration between adjacent voltage pulses within a series of voltage pulses.

In some embodiments, the width, or duration, of an applied voltage pulse may be within a range of about 20 to about 30 microseconds. In some embodiments, the width, or duration, of an applied voltage pulse may be about 25 microseconds.

In some embodiments, the predetermined number of voltage pulses within a series of voltage pulses may be within the range of 5 voltage pulses to 20, or more, voltage pulses. In some embodiments, the number of voltage pulses within a series of voltage pulses may be about 10 voltage pulses.

In some embodiments, a predetermined maximum number of voltage pulses that are allowed for any one of the catheter assemblies 14 may be within the range of 50 voltage pulses to 300 or more voltage pulses.

Each of the control data comprising at least the predetermined starting voltage, the upper voltage threshold, the width or duration of the generated and/or applied voltage pulse, the predetermined pause between adjacent voltage pulse series, the time gap between adjacent voltage pulses within a voltage pulse series, the number of generated voltage pulses required before increasing voltage, the magnitude of an increase in voltage, and/or the maximum number of voltage pulses allowed for a given catheter assembly 14 may be stored in, and executed by, the control system 22.

In other embodiments, as will be discussed below, an EPROM device as is well known in the art, may be connected to, or associated with, a handle that is in operative association with the catheter assembly. In these embodiments, the control data comprising at least the predetermined starting voltage, the upper voltage threshold, the predetermined pause between adjacent voltage pulse series, the time gap between adjacent voltage pulses within a voltage pulse series, and/or the maximum number of voltage pulses allowed for a given catheter assembly 14 may be stored in the EPROM which is operative association with the control system 22. The EPROM may allow identification of a particular catheter assembly 14 which may require its own individualized set of control data, thus allowing highly flexible operation.

The control system 22 in the various embodiments may monitor data corresponding with the control data and, if the monitored data is not compliant with a stored range of values, or a within a threshold value, the control system 22 may notify the operator and may, in some embodiments, allow no further generation of voltage pulses.

With continued reference to FIGS. 1, 2, 3A and 3B, portions of the illustrative electric pulse generation system 20, including portions of the IVL control system 22, are disclosed herein including various features and/or circuitry which may be implemented as portions of circuitry 28, although in some embodiments, such systems 20, 22 may share components and/or have isolated components as applicable, for example, such that circuitry 28 is intended to be diagrammatic and may also represent circuitry embodied by system 20 alone as applicable. In the illustrative embodiment, the IVL control system 22 includes an adjustable energy storage system, illustratively an exemplary capacitance system, 112 for selective adjustment of the energy storage capacity or magnitude applied for providing electrical energy to the electrodes. We refer hereinafter to energy storage system 112, illustrated by but certainly not limited to, the capacitance system.

The energy storage system 112 receives charge electrical energy from a power source of the electric pulse generation system 20. The energy storage system 112 provides discharge electrical energy to the electrodes (e.g., illustratively via VCAP2 and grounding), as discussed in additional detail herein.

The adjustable energy storage system 112 illustratively includes a number, e.g., one or more, of energy storage units, exemplified as individual capacitors 114, defining an energy storage network. In the illustrative embodiment, each energy storage unit 114 may be sized to have the same energy storage capacity and may be arranged for parallel connection with the other energy storage unit(s) in the energy storage network, although in some embodiments, any suitable size and/or arrangement of the energy storage unit(s) may be provided to support variable energy storage for IVL therapy. A relay system 116 may be arranged in connection with at least some of the energy storage elements 114 of the network. The relay system 116 comprises one or more relays for selectively connecting energy storage element(s) 114 together to receive charge and to discharge electrical energy to the electrodes.

In the illustrative embodiment, the relay system 116 includes an engaged arrangement in which all energy storage elements 114 of the network are connected for IVL use. Energy storage element(s) 114, when connected for IVL use, may be connected with other portions of the electric pulse generation system 20 to exchange electrical energy under other control operations. For example, under typical charge control operations, energy storage elements 114 connected for IVL use by relay system 116 may receive charge from power supply, and/or under typical discharge control operation, energy storage elements 114 connected for IVL use by relay system 116 may provide discharge energy to the electrodes 18. Accordingly, it can be appreciated that the relay system 116 can selectively connect all energy storage element(s) 114 for use in the IVL therapy, to provide a maximum energy storage magnitude.

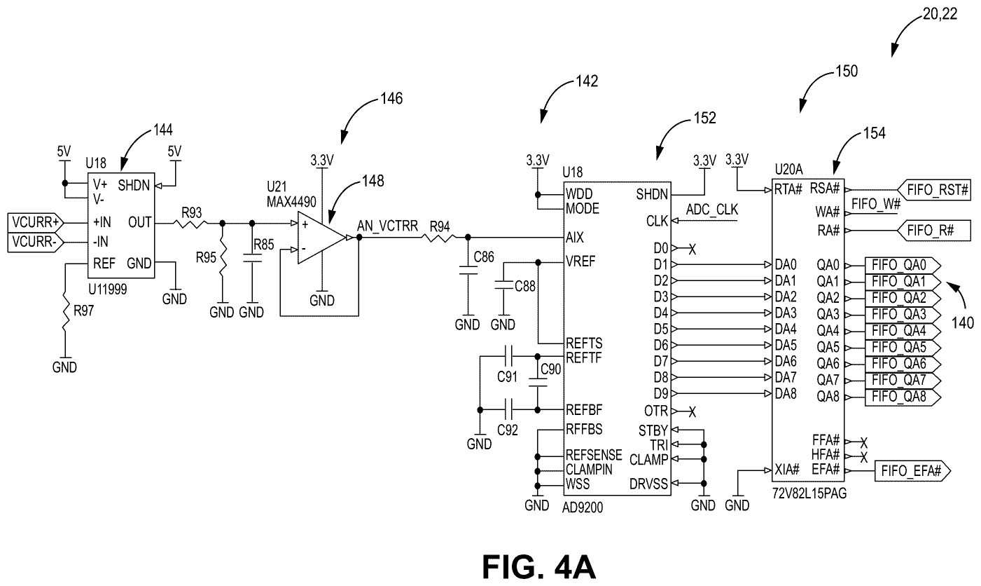

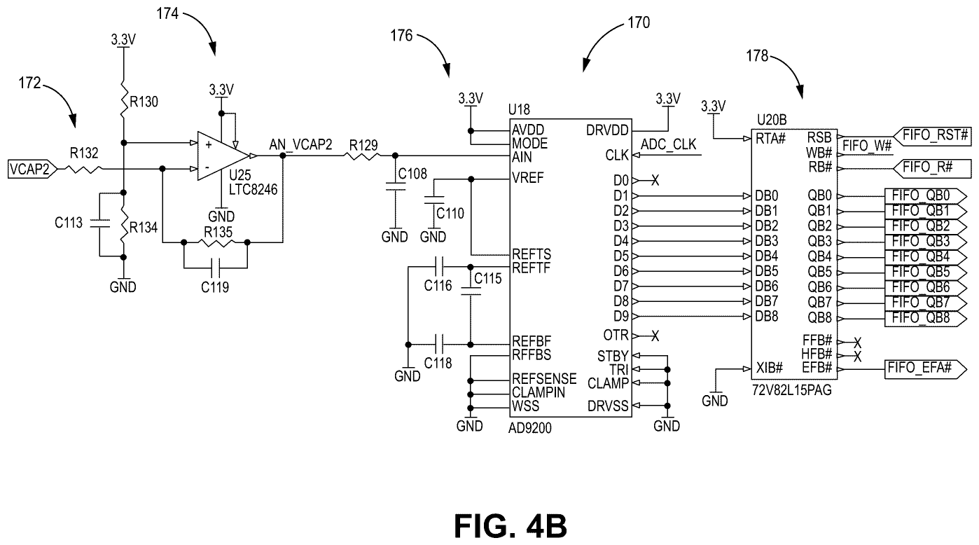

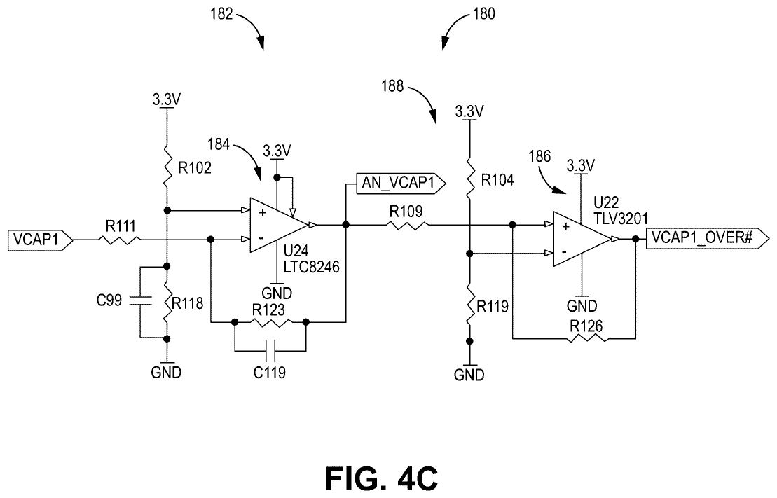

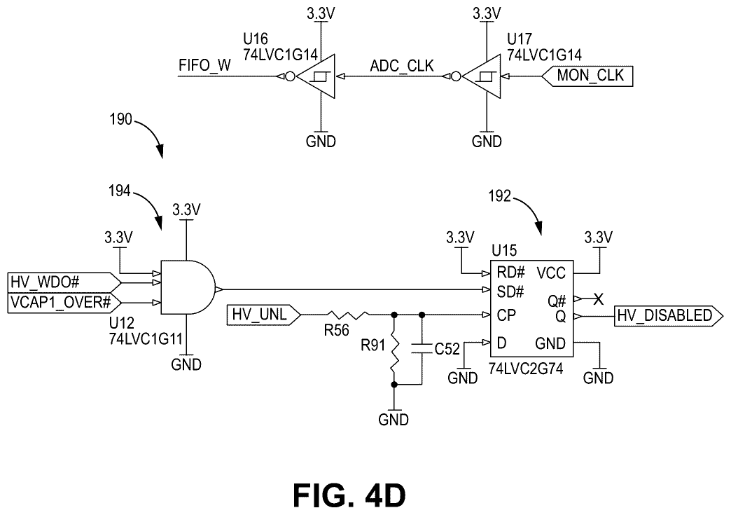

Additionally, the relay system 116 includes a disengaged arrangement in which fewer than all energy storage elements 114 of the network are connected for IVL use as suggested in FIGS. 4A-4D. A number of energy storage elements 114, illustratively two energy storage elements, are rendered disconnected from the other energy storage element(s) by disengagement of the relay system 116 for ease of description but without limitation. Energy storage elements 114 disconnected for IVL use by relay system 116 cannot receive charge from power supply, and/or under typical discharge control operation, energy storage elements 114 disconnected for IVL use by relay system 116 cannot provide discharge energy to the electrodes 18. Energy storage elements which are disconnected for use in IVL therapy may be discharged apart from the electrodes (e.g., for safe reduction of stored power), illustratively via diode 118 arranged in parallel with the relay system 116.

It can be appreciated that by adjustment of the energy storage capacity available to provide electrical energy to the electrodes, the total amount of energy provided to the electrodes can be controlled. The stored energy can be indicated according to

$$\frac{1}{2} C * {V}^{2} ,$$

where V represents the voltage and C represents the capacitance, such that applied electrical energy is directly proportional to the amount of energy connected in use. Accordingly, by selectively engaging the relay system 116 to close the circuit and connect the disconnected energy storage element(s), the energy storage capacity of the IVL system can be adjusted to provide variable energy levels to the electrodes. Although the illustrative embodiment includes relay control for connection and/or disconnection of a pair of energy storage elements, any suitable number of relays and/or energy storage elements may be applied to provide adjustable energy storage capacity for IVL therapy.

In the illustrative embodiment, the IVL control system 22 is configured to govern the applied stored energy. The IVL control system 22 illustratively determines the amount of stored energy to be applied, and upon determination that a change in the energy storage magnitude is desired, the IVL control system 22 operates the relay system 116 accordingly. For example, the IVL control system 22 may determine that one voltage pulse desires and/or requires lower energy storage magnitude and may communicate to operate the relay system 116 in the disengaged arrangement.

For one or more subsequent voltage pulses, the IVL control system 22 may determine that another voltage pulse desires and/or requires greater energy storage magnitude and may communicate to operate the relay system 116 in the engaged arrangement. For one or more further subsequent voltage pulses, the IVL control system 22 may determine that lower energy storage magnitude is again desired and/or required and may return the relay system 116 to the disengaged arrangement. Accordingly, the IVL control system 22 may operate the relay system 116 as needed to provide adjustable energy storage magnitudes for any given voltage pulse within a series of voltage pulses.

The applied energy storage may be adjusted on an ongoing basis, for example, for any given pulse. In practice, adjusting the energy storage capacity or magnitude may be conducted in conjunction with the level of voltage to be applied and/or in consideration of other aspects of power, efficiency, and/or technique. Moreover, under the lifetime of applied devices and systems, typical wear to components can alter the electrical and/or physical characteristics thereof, which may benefit from adjustment to the energy storage that is applied. For example, even small wear to electrodes can vary the spacing (gap) between a set of electrodes which can alter the conditions of the arc between electrodes. Accordingly, adjustable energy storage magnitude can accommodate variations in the electrodes and/or or portions of the discharge system under repeated use, whether in individual therapy sessions or otherwise.

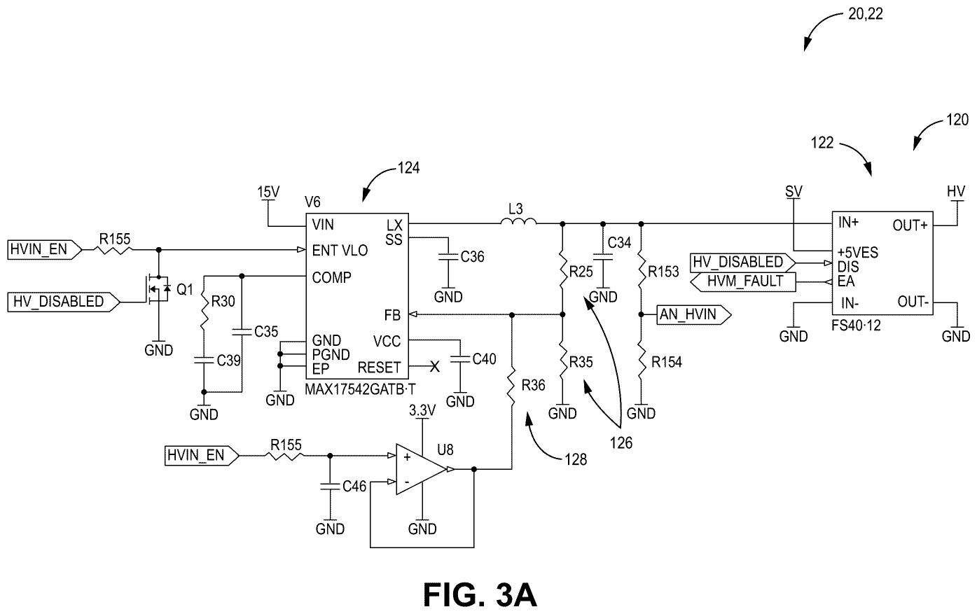

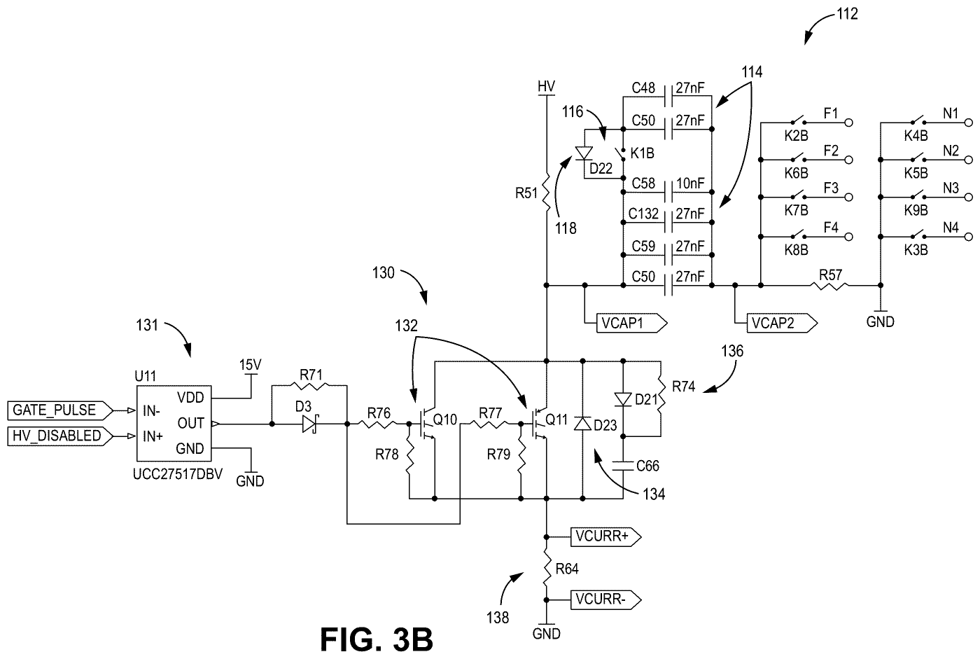

With continued reference to FIGS. 3A and 3B, embodiments of the disclosure are directed to systems and methods for adjusting the voltage provided to charge the energy storage system 112 as charge voltage. The charge voltage is illustratively provided as input to the energy storage system 112 as energy accumulation for discharge to generate a voltage pulse, controlled for variable charge. The charge voltage is illustratively controlled by a charge control system 120 of the IVL control system 22.

In the illustrative embodiment, the charge control system 120 provides accurate control of charge voltage via high frequency switched control signals from the processor 24. The switched control signal (“HVIN_VSET”), illustratively embodied as a pulse width modulated (PWM) signal, is amplified for control of high voltage supply. A high voltage DC/DC converter system 122 receives indication of the switch control signal within the range of about 0 V to about 12 V, and provides a corresponding charge voltage illustratively within the range of about 0 V to about 4000 V.

In the illustrated embodiment, the charge control system 120 includes a buck regulator system 124 for conditioning the low voltage power. The buck regulator system 124 is illustratively embodied as an integrated circuit (IC) to provide signal conditioning with low pass filtering and buffering of the PWM signal. The buck regulator system 124 receives a conditioned PWM signal with feedback for providing controlled low voltage power to the converter system 122 for applying high voltage power.

Resistors 126 can scale down the feedback voltage appropriately for the IC operation, to provide a variable feedback voltage to the buck regulator system 124 within a range of about 0 V to about 3.3 V at the additional resistor 128. As the duty-cycle of the PWM signal increases from zero to 100 percent, the filtered signal increases from zero volts to 3.3 volts, which can increase the current provided to the feedback network, and can require less voltage to achieve regulation, for example, at resistors, inductors, and/or capacitors arranged between the buck regulator system 124 and the converter system 122.

Referring still to FIGS. 3A and 3B, embodiments of the disclosure are directed to systems and methods for controlling the active time for discharge voltage pulses provided to the electrodes. A switching signal (e.g., “GATE_PULSE”) is provided by the processor 24 for high voltage switching via low voltage signaling. In the illustrative embodiment, the switching signal is applied to precisely activate a discharge switch system 130. The discharge switch system 130 is illustratively embodied to implement a driver 131 and semiconductor devices 132 as gate switches.

The gate switches 132 are illustratively embodied as insulated gate bipolar transistors (IGBT) having n-type gate-controlled arrangement. On active state of the switching signal to the driver 131, the gate switches 132 are activated into their conducting state to communicate discharge of energy from the energy storage system 112 to the electrodes. On inactive state of the switching signal the gate switches 132 are deactivated into their non-conducting states, blocking discharge of the energy storage system 112 to the electrodes.

In the illustrative embodiment, the gate switches 132 are arranged as active high devices, although in some embodiments, may be implemented as active low devices. Gate-controlled, active high IGBTs can provide precision control of discharge from the energy storage system 112, but may be implemented by any suitable manner including by other suitable semiconductors (e.g., p-type, FET, etc.) and/or other control designs (e.g., collector, emitter control, etc.).

In the illustrative embodiment, the discharge switch system 130 includes an anti-parallel diode 134 arranged to reduce reverse-voltage stresses on the gate switches 132. A disable signal (“HV_DISABLED”) is provided to the driver 131 which permits discharging of energy to the electrodes on inactive (low) signal, and may activate (high) to disable high voltage discharge under direction of the processor 24 and/or other safety systems. The snubber system 136 is illustratively embodied as a resistor-capacitor-diode (RCD) snubber network arranged to reduce voltage transients that may exceed rated voltages of various high-voltage components.

Referring now to FIGS. 4A-4D, the IVL control system 22 illustratively includes electrical power monitoring system 140. The electrical power monitoring system 140 is configured to conduct monitoring of various parameters of electrical power of the IVL device and systems, illustratively including sensing of current and voltage delivered to the electrodes, and voltage of the adjustable energy storage system 112.

The electrical power monitoring system 140 illustratively includes a current monitoring system 142. The current monitoring system 142 is embodied to sense the current delivered to the electrodes for a given voltage pulse. As discussed in additional detail herein, the current delivered to the electrodes can be considered in determining the power characteristics for subsequent voltage pulses.

In the illustrative embodiment as shown in FIGS. 4A-4D, the current monitoring system 142 receives indication of the voltage levels applied in each voltage pulse for determining current delivered to the electrodes. Returning briefly to FIGS. 3A and 3B, a shunt resistor 138 is arranged within the high-voltage current path establishing a proportional voltage (e.g., “VCURR+”, “VCURR−”). The proportional voltage is communicated to the current monitoring system 142 as shown in FIGS. 4A-4D.

A chip comprising an amplifier 144 is arranged to scale-up the proportional voltage, and provides the analog result to a conditioning network 146, embodied to include a resistor-capacitor network for scaling and/or filtering. The conditioning signal is buffered by a buffering amplifier 148, the output of which is provided to an analog-to-digital conversion (ADC) system 150 for digital conversion.

The ADC system 150 illustratively includes a converter 152 and memory 154. In the illustrative embodiment, the converter 152 provides digital output from the analog input, and the memory 154 is embodied as a first-in-first-out (FIFO) device for intermediate storage of digital outputs. The memory 154 illustratively receives the same clock signal driving the converter 152, to allow quick sampling of a number of measurement points with low-jitter. Memory outputs are provided to the processor 24 for consideration in overall IVL therapy control.

The IVL control system 22 illustratively includes a current monitoring system which compares the output signal (“VCURR”) generated by the chip comprising an amplifier 144 with a threshold value. The threshold value is embodied to be generated by a variable duty cycle PWM signal (“ISNS_ISET”) from the processor 24. The PWM signal can be low-pass filtered and/or buffered before delivery to a comparator. Responsive to the measured current exceeding the threshold value, the current monitoring system can assert an error signal (e.g., “ISNS OVER #”) to avoid overcurrent conditions.

The electrical power monitoring system 140 illustratively includes a voltage monitoring system 170. The voltage monitoring system 170 is embodied to sense the voltage between the set of electrodes. As discussed in additional detail herein, the voltage between the electrodes for a given pulse can be considered in determining the power characteristics for subsequent voltage pulses.

In the illustrative embodiment, the voltage monitoring system 170 includes a resistor network 172 arranged to attenuate the (switched) voltage of one of the electrodes of a set (e.g., “VCAP1”). The attenuated signal is provided to an op amp network 174 for filtering and offsetting for output to digital conversion. The output from the op amp network 174 is provided to the ADC conversion system 176 for digitization, including storage in FIFO memory 178 for access by the processor 24.

The electrical power monitoring system 140 may illustratively include an energy storage capacity voltage monitoring system 180 configured for monitoring the voltage within the adjustable energy storage system 112. Monitoring the voltage of the energy storage system 112 can allow determination of the stored energy of the energy storage system 112. Moreover, comparison of the stored energy of the energy storage system 112 before and after discharge can provide indication of the total energy delivered during a given discharge cycle. Such total energy data can be considered to increase confidence in determining whether a sufficient spark has been generated for IVL therapy.

In the illustrative embodiment, the voltage monitoring system 180 includes a voltage limiting system configured to monitor net voltage of the energy storage system 112 during charging. In the illustrative embodiment, voltage monitoring is discussed relative to the connected energy storage elements 114, more specifically, those energy storage elements which are connected to provide controlled discharge energy for IVL therapy, and not energy storage elements which are disconnected via the relay system 116 if any.

The voltage monitoring system 180 receives indication of the voltage of the energy storage system 112 during charging (“VCAP1”). The system 180 illustratively includes an amplifier configuration 182 comprising an amplifier 184 and a comparator 186. The comparator 186 is illustratively arranged to compare to the voltage with a fixed voltage, and to responsively trigger a signal (e.g., “VCAP1_OVER #”) when the voltage of the energy storage system 112 exceeds the fixed voltage. In the illustrative embodiment, the fixed voltage is embodied as setpoint generated by a resistor-resistor-capacitor network 188 above normal operation, but before damage will occur to various HV components.

An intermediate voltage of this circuit (e.g., “AN_VCAP1”) can be used to monitor progress during the charging cycle of the energy storage system 112. The intermediate voltage illustrative represents a heavily attenuated indication of the high voltage provided by the energy storage system to the electrodes (“VCAP_1”). Such attenuated signal can allow monitoring of high voltage systems while handling lower voltage indications thereof.

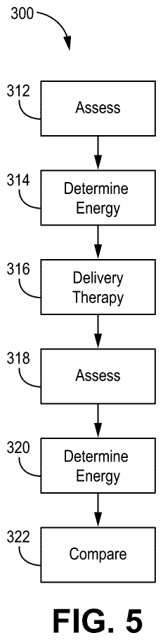

Referring now to FIG. 5, and with continued reference to FIGS. 1 and 3, the IVL system 12 can consider energy of the energy storage system 112 in operation. By monitoring energy of the energy storage system 112 before and after a discharge event, indication of the generation (and/or sufficiency) of spark can be determined as discussed in additional detail regarding the illustrative embodiment with reference to operation 300 concerning boxes 312 through 322.

In box 312, assessment of the energy storage system 112 is conducted. In the illustrative embodiment, the assessment includes determination of a voltage of the energy stored by the energy storage system 112. As mentioned above, the voltage monitoring system 180 can monitoring voltage of the energy storage system 112, for example, via the voltage limiting system during charging. In some embodiments, the assessment may include determination of any other suitable parameters to support energy monitoring of the energy storage system 112.

In box 314, the energy of the energy storage system 112 is determined. In the illustrative embodiment, the energy of the energy storage system 112 is determined based on the measured voltage according to

$$\frac{1}{2} C * {V}^{2} .$$

Accordingly, the processor 24 can compute the current energy of the energy storage system 112, including the energy stored just before discharge of energy to the electrodes.

In box 316, IVL therapy can be attempted. In the illustrative embodiment, a voltage pulse can be delivered to the electrodes. The voltage pulse can be applied according to the control arrangement as mentioned herein, for example, based on a determined duration in a control sequence.

In box 318, assessment of the energy storage system 112 is conducted. Assessment of the energy storage systems 112 in box 218 is embodied as occurring immediately after attempted IVL therapy in box 216 to provide an indication of the energy state of the energy storage system 112 immediately after (attempted) discharge to the electrodes. In the illustrative embodiment, the assessment includes determination of a voltage of the energy stored by the energy storage system 112, embodied as conducted by the voltage monitoring as mentioned above, although in some embodiments, assessment of the energy storage system 112 in box 318 may differ from box 312 in methodology and/or practice.

In box 320, the energy of the energy storage system 112 is determined. In the illustrative embodiment, the energy of the energy storage system 112 is again determined based on the measured voltage according to

$$\frac{1}{2} C * {V}^{2}$$

just as in box 214, yet after attempted delivery of IVL therapy. In some embodiments, determining energy of the energy storage system 112 in box 320 may differ in methodology and/or practice from that in box 214. Accordingly, the processor 24 can compute the current energy of the energy storage system 112, including immediately after (attempted) discharge of energy to the electrodes.

In box 322, comparison between energy determinations is conducted. The amount of energy determined within the energy storage system 112 in box 314 is illustratively subtracted from the amount of energy determined within the energy storage system 112 in box 320, such that the result represents the amount of energy discharged from the energy storage system 112 under a single attempt to deliver IVL therapy.

The amount of energy discharged can be considered to determine whether a spark (or sufficient spark) has occurred such that IVL therapy occurs. In the illustrative embodiment, a threshold energy discharge represents a discharged energy level which confidently indicates spark sufficient for IVL has occurred. Accordingly, in box 322, comparing the stored energy levels before and after discharge to determine whether the threshold energy discharge has been achieved can indicate spark for IVL therapy.

In the illustrative embodiment, and with reference to FIG. 2, the threshold energy discharge is a fixed predetermined value, for example, 600 millijoules (e.g., 3700 V, 90 nanofarad). However, in some embodiments, the threshold energy level for a given cycle may be determined based on the number of previous cycles in a therapy session, the number of cycles maintaining present settings (e.g., through box 46 of FIG. 2) in a therapy session, the number of particular successive cycles (e.g., number of successive cycles through box 46 or box 48 or box 52 or box 54 or box 58), the patient characteristics, environmental conditions (e.g., location within patient's body such as above the knee or above the knee), procedural approach, and/or product cycle lifetime (i.e., operable age), among other aspects.

Comparison of energy levels of the energy storage system 112 which indicates a spark for IVL therapy has occurred can responsively cause further therapy at the same duration, energy level, threshold characteristic, and/or threshold adjusted for other parameters. Comparison of energy levels of the energy storage system 112 which indicates a spark for IVL therapy has not occurred can cause adjustment to the duration and/or energy level applied, for example, as discussed concerning control operation 38.

In some embodiments, the threshold current value can be applied together with the threshold energy discharge, such that either threshold can individually indicate a spark for IVL therapy. In some embodiments, both thresholds may be required to be met to indicate a spark for IVL therapy.

Consideration of the energy states of the energy storage system 112 can provide desirable monitoring of IVL operations. For example, such monitoring can be less intrusive by reducing the need for direct measurements at the electrodes. Moreover, in high power applications, reliable consideration of the energy states can promote confidence over mere direct measurement in unpredictable high energy arc scenarios.

The IVL control system 22 illustratively includes an external watchdog system configured to assist in safe operation. The watchdog system includes an integrated circuit configured to trigger an error under lack of a timely toggled input signal to ensure appropriate high voltage operation. In some embodiments, the watchdog system may be formed externally including processor, memory, and/or circuitry distinct from or shared with the IVL control system 22.

Returning to FIGS. 3A and 3B, in the illustrative embodiment, the IVL control system 22 includes an umbrella monitoring system 190 configured to assist in safe operation. The umbrella monitoring system 190 illustratively includes a flip flop 192 and logic gate 194 for consideration of monitoring signals. The logic gate 194 is arranged to receive monitoring signals, embodied as energy storage system overvoltage (“VCAP1_OVER #”) from the voltage monitoring system 180, high voltage warning (“HV_WDO #) from the watchdog system, and in some embodiments, may receive overcurrent from the current monitoring system (“ISNS_OVER #”).

The logic gate 194 is embodied as an AND gate and the flip flop 192 embodied as an asynchronous D-flip flop, such that activation signals from the gate 194 which last longer than the minimum clock pulse width of the flip flop 192 cause an assertion of outputs to disable high voltage output (e.g., “HV_DISABLED”), but activation signals from the gate 194 shorter than the minimum clock pulse width of the flip flop 192 do not raise disabling outputs from the umbrella monitoring system 190.

Assertion of the signal to disable high voltage output (“HV_DISABLED”) is illustratively provided to the discharge switch system 130 to disable voltage pulse switch activation to the electrodes. In the illustrative embodiment, the disabling output signal is provided to the driver 131 and indirectly to alter on/off operation of the gate switches 132. Such disabling output signal is illustratively provided to low-voltage supplies, e.g., buck regulator system 124, and high voltage modules, e.g., converter system 122.

Accordingly, the logic gate 194 receives monitoring signals discussed above comprising: (1) energy storage system overvoltage (“VCAP1_OVER #”) from the voltage monitoring system 180, (2) high voltage warning (“HV_WDO #) from the watchdog system, and in some embodiments, (3) may receive overcurrent from the current monitoring system (“ISNS_OVER #”). These monitoring signals ae also connected to a three-input AND logic gate [U12], which is upstream of a D flip-flop with asynchronous set and reset functionality [U15], so that any signal that asserts for longer than the minimum pulse width of the flip-flop [U15] will cause its outputs [HV_DISABLED, HV_DISABLED #] to assert. These signals travel downstream and inhibits the operation of the gate driver [U11], variable low-voltage supply [U6], high-voltage module [U7], and slightly changes the turn-on and turn-off operation of the switching devices [Q10, Q11] via transistors [Q12, Q13].” This system allows any one of monitoring signals to disable the output of the system if asserted longer than an established duration.

Within the present disclosure, the ability to run off of AC mains or battery DC can afford versatility of power and control for IVL therapy. Unlike known IVL systems, certain embodiments within the present disclosure can avoid sitting idle until fully (or substantially) recharged in order to be applied in IVL therapy, for example, if insufficiently charged by the time IVL therapy is desired. Accordingly, such costly delays, or interruptions, in procedure can be avoided with embodiments of the present disclosure. The electric pulse generation system 20 illustratively includes a battery power storage system, and is configured to selectively charge the energy storage system 112 from battery stored energy only, from the battery power storage system while connected to mains, such as outlet power, or directly from DC power converted from the AC mains without the battery power storage system. When plugged in to AC mains, regulated DC power is delivered directly to the high voltage systems. In operational states in which the current demand for IVL operations is high, the battery power storage system charge current can be reduced to allow for higher IVL operations system current. When not plugged in to AC mains, battery power can be delivered directly to energy storage system 112. In the illustrative embodiment, systems and devices of power management, including for example, inverters, conditioners, power storage devices, and/or related aspects may be comprised by the electric pulse generation system 20 to provide applicable power to the IVL control system 22.

Examples of suitable processors may include one or more microprocessors, integrated circuits, system-on-a-chips (SoC), among others. Examples of suitable memory, may include one or more primary storage and/or non-primary storage (e.g., secondary, tertiary, etc. storage); permanent, semi-permanent, and/or temporary storage; and/or memory storage devices including but not limited to hard drives (e.g., magnetic, solid state), optical discs (e.g., CD-ROM, DVD-ROM), RAM (e.g., DRAM, SRAM, DRDRAM), ROM (e.g., PROM, EPROM, EEPROM, Flash EEPROM), volatile, and/or non-volatile memory; among others. Communication circuitry 58 includes components for facilitating processor operations, for example, suitable components may include transmitters, receivers, modulators, demodulators, filters, modems, analog/digital (AD or DA) converters, diodes, switches, operational amplifiers, and/or integrated circuits. In some embodiments, memory 26 may represent one or more memory devices operable for IVL therapy operation. For example, each memory (e.g., 154, 178) may be included as part of memory 26, shared, or isolated therefrom.

Within the present disclosure, consideration of a set of discharge electrodes has been discussed in the context of a pair of electrodes, one of which may serve as a cathode and the other of which may serve as an anode, at a given instance. However, the number of electrodes in a set may be greater than a pair, for example, including one or more cathodes communicating with one or more anodes. Additionally, devices, systems, and methods within the present disclosure may include more than one communicating group of electrodes, whether electrically arranged serially, parallel, or independently from each other.

Power control operations disclosed herein may be applied equally, simultaneously, and/or sequentially to individual sets or groups of electrodes, in a given IVL therapy cycle. For example, threshold current value may be applied collectively to all deployed electrodes or to individual groups or sets of electrodes. Determinations made with respect to power control may be applied equally to related electrodes, or may be individualized to groups or sets of electrodes, in a given IVL therapy cycle. Within the present disclosure, supporting components, such as power supplies, sensors, and other implementing structures and/or features for performing IVL operations as disclosed herein are embodied as sub-portions of the electric pulse generation system 20 and/or IVL control system 22, for example, as parts of circuitry and/or instructions.

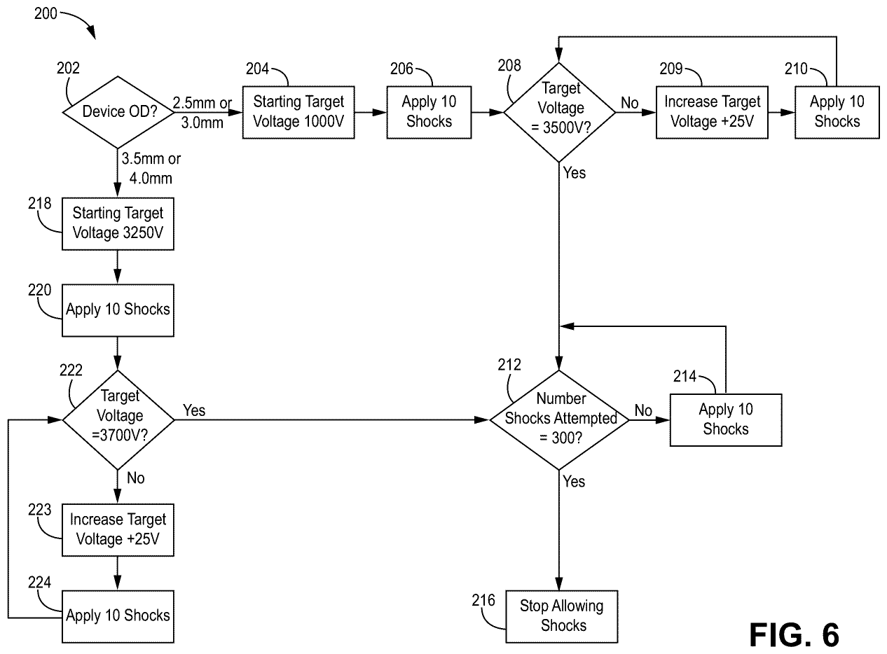

Referring now to FIG. 6, an exemplary flow diagram is shown concerning another embodiment of a control 200 in operation of an embodiment of an IVL system, specifically the number of voltage pulses generated and the magnitude of the generated pulses. It is understood that the control 200 may be combined with aspects of the control system and method embodiments described above in relation to FIGS. 1-5.

Control operations of control 200 may be governed by the electric pulse generation system 20, and illustratively by the IVL control system 22 discussed above in connection with FIG. 1. As discussed further herein, control system 22 may control the number of generated voltage pulses in a series (or a plurality of series) of voltage pulses. An acceptable voltage window comprises a predetermined starting voltage magnitude and a predetermined upper voltage magnitude. IVL control system further comprises a predetermined voltage magnitude for incremental increasing of voltage magnitude after each series of voltage pulses if the magnitudes of the executed voltage pulses is within the acceptable voltage window. Separate sets of predetermined control data may be provided in control system 22 for IVL systems comprising balloons that are of identifiable characteristics such as, without limitation, different sizing, e.g, 2.5 mm, 3.0 mm, 3.5 mm and/or 4.0 mm.

An exemplary embodiment of an IVL system may comprise a 2.5 mm or a 3.0 mm balloon, wherein the control system 22 comprises control data comprises an exemplary starting target voltage of 3000V (predetermined lower voltage threshold), voltage pulse series comprising an exemplary 10 pulses, and an exemplary incremental voltage increase of 25V if the voltage pulse series magnitudes are less than an exemplary upper voltage threshold of 3500V. As the skilled artisan will recognize, the incremental voltage increase may comprise any voltage magnitude, including without limitation within 1V to 250V. An exemplary voltage increase may comprise 25V, but may be greater or less than 25V in certain embodiments. As the artisan will recognize, the predetermined starting voltage may be less than 3000V and the predetermined upper voltage threshold may be greater than 3500V. Thus, an exemplary starting voltage may, without limitation, comprise 2500V and an exemplary upper voltage threshold may comprise 4100V. In other embodiments, an exemplary predetermined starting voltage maybe greater than 3000V and an exemplary upper voltage threshold may be greater than 3250V.

Another exemplary embodiment may comprise a 3.5 mm or a 4.0 mm balloon, wherein the control system 22 comprises control data comprises an exemplary starting target voltage of 3250V (predetermined lower voltage threshold), voltage pulse series comprising 10 pulses, and an incremental voltage increase of 25V if the voltage pulse series magnitudes are less than an exemplary predetermined upper voltage threshold of 3700V.

With continued reference to FIG. 1, and in some embodiments FIG. 2, FIG. 6 illustrates initiation of a voltage pulse generation and control system 200 that begins with box 202 which requires determination of a particular balloon characteristic of interest, for example the outer diameter (“OD”) of the IVL system's balloon. In a first embodiment, if the balloon's outer diameter is, e.g., 2.5 mm or 3.5 mm, then in box 204 the starting voltage is set to 3000V, also referred to as the predetermined lower voltage threshold of the acceptable voltage magnitude window). The IVL therapy is initiated in box 206 by application of a series of voltage pulses (or shocks) from the electric pulse generation system 20 wherein each voltage pulse travels to the electrodes 18 within the balloon 16. If, in box 208, the target voltage magnitude is not at the predetermined upper voltage threshold, e.g., 3500V, then as in box 109, the target voltage is increased by an exemplary 25V (from 3000V to 3025V) and another series of voltage pulses (in this case 10 pulses) is executed, as in box 210 at 3025V. This process continues with cycling between boxes 208, 209 and 210 until the target voltage is at 3500V. When the target threshold voltage, or predetermined upper voltage threshold, is reached, and/or in some embodiments a predetermined maximum or desired number of voltage pulses, e.g, 300 pulses (or shocks) have been generated, then as in box 212, control system 22 determines if the number of generated voltage pulses (or shocks) in the plurality of series of voltage pulses has reached a maximum, or desired, number of pulses, e.g., 300 voltage pulses. If the maximum or desired, e.g., 300, voltage pulse threshold has not been reached, then as in box 214 another series of, e.g., 10, voltage pulses (or shocks) are applied. When the maximum or desired, e.g., 300 voltage pulse threshold has been reached, then as in box 216, additional voltage pulses (or shocks) are not allowed.

The predetermined maximum number of voltage pulses in various embodiments of the present disclosure may be within the range of 10-300 voltage pulses. The exemplary embodiments discussed herein comprise a predetermined maximum number of voltage pulses equal to 300 pulses. In other embodiments, the maximum number of voltage pulses may be greater than 300 pulses.

In a second embodiment, with continued reference to FIG. 1, and in some embodiments, FIG. 2, if the balloon's outer diameter is determined in box 202 to be, e.g., 3.5 mm or 4.0 mm, then the electric pulse generation system 20 initiates therapy at box 118 the starting voltage is set to 3250V, also referred to as the predetermined lower voltage threshold of the acceptable voltage magnitude window). The IVL therapy is initiated in box 220 by application of a series of voltage pulses from the electric pulse generation system 20 wherein each voltage pulse travels to the electrodes 18 within the balloon 16. If, in box 222, the target voltage magnitude is not at the predetermined upper voltage threshold, e.g., 3500V, then as in box 223, the target voltage is increased by an exemplary 25V (from 3250V to 3275V) and another series of voltage pulses (in this case 10 pulses) is executed, as in box 224, at 3275V. This process continues with cycling between boxes 222, 223 and 224 until the target voltage is at 3700V. When the target threshold voltage, or predetermined upper voltage threshold, is reached, and/or in some embodiments a maximum or desired number of pulses, e.g, 300 pulses (or shocks when applied to the one or more pairs of spaced-apart electrodes) have been generated, then as in box 212, control system 22 determines if the number of generated voltage pulses (or shocks) in the plurality of series of voltage pulses has reached a maximum, or desired, number of pulses, e.g., 300 voltage pulses. If the maximum or desired, e.g., 300, voltage pulse threshold has not been reached, then as in box 214 another series of, e.g., 10, voltage pulses (or shocks) are applied. When the maximum or desired, e.g., 300 voltage pulse threshold has been reached, then as in box 216, additional voltage pulses (or shocks) are not allowed.

Alternatively, the physician conducting the IVL therapy according to the voltage pulse generation and control system 200 may determine that the therapy is complete at a point during execution of the therapy. If the therapy is determined to be completed, then the physician may terminate the process of the voltage pulse generation and control system 200 at any point.

In some embodiments, the voltage pulse generation and control system 200 may comprise modification of the duration of applied voltage within, or across, one or more of the plurality of series of voltage pulses in accordance with the embodiments discussed above in connection with FIG. 2. For example, duration may be increased, or decreased, by a predetermined duration interval, illustratively embodied as a fixed value, e.g., 0.5 microseconds. In some embodiments, the predetermined interval for given cycle may be determined based on factors such as the number of times therapy cycles have ensued previously during the therapy session (e.g., number of series of voltage pulses that have been executed by proceeding through boxes 206, 208 and 210, or 220, 222 and 224), the patient characteristics, environmental conditions, procedural approach, and/or product cycle lifetime (i.e., operable age), among other aspects. In some embodiments, the predetermined duration interval for a given cycle may be varied by predetermined rate of change, for example, by percentage gain or loss by cycle.

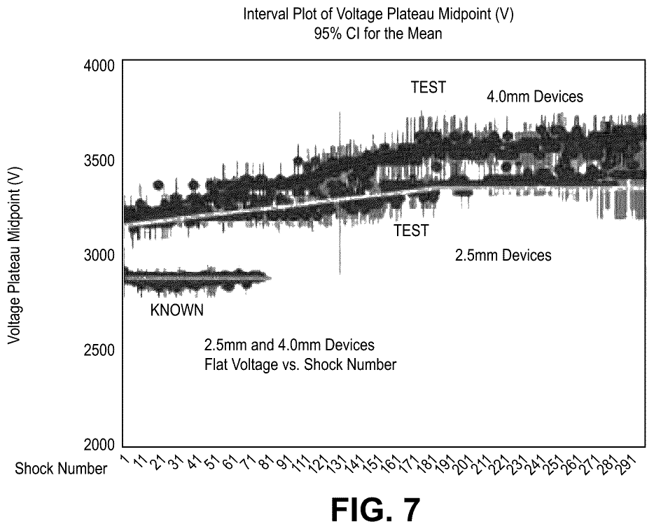

FIG. 7 presents a graphic comparison of known IVL devices comprising 2.5 mm and 4.0 mm OD balloons (KNOWN) with IVL devices according to the present disclosure and comprising 2.5 mm and 4.0 mm OD balloons (TEST).

The KNOWN devices permit 80 voltage pulses or shocks to be generated. The TEST devices generated 300 voltage pulses or shocks. During the comparative testing, for each voltage pulse, the voltage peak magnitude was obtained and plotted for each tested device. The KNOWN devices provide a relatively flat or constant voltage for each voltage pulse or shock number and begin at a lower voltage magnitude than the TEST devices. The TEST devices were operated and controlled in accordance with the disclosed embodiments herein.

In contrast, each of the 2.5 mm and 4.0 mm TEST devices begin at a higher voltage magnitude than the KNOWN devices. The 2.5 mm TEST device begins at a lower voltage than does the 4.0 mm device. As illustrated, both the 2.5 mm and the 4.0 mm TEST device voltage (lower data cluster) rise slowly over the generated voltage pulses, plateauing at approximately 180 pulses, thereafter remaining substantially flat or constant. In each case, the average voltage of the 4.0 mm TEST device is greater than that of the 2.5 mm TEST device.

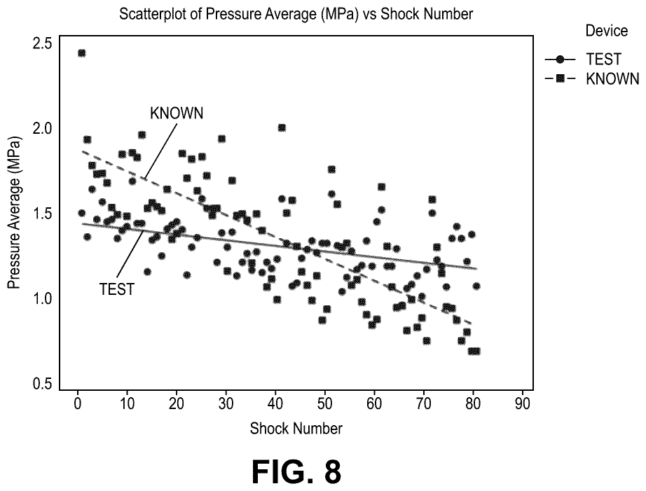

FIG. 8 illustrates a comparison of the TEST and KNOWN IVL devices and a subset of the data of FIG. 7, i.e., comparing the peak pressure outputs generated by the first 80 voltage pulses for each device type. Here, the voltage pulse (or shock number) is compared with the peak pressure generated during each voltage pulse. The KNOWN data with a constant voltage in each voltage pulse presents (dashed line) a relatively severe decrease in pressure output as the voltage pulses progress over time. In contrast, the TEST data (solid line) obtained using the voltage and pulse generation algorithm of FIG. 2, presents a pressure output line that decreases at a much smaller angle or slope. Thus, the pressure output of the TEST IVL devices provides a more stable or constant pressure output than the KNOWN IVL devices. The KNOWN IVL devices have a pronounced pressure output decay as the voltage pulses progress. More specifically, the TEST IVL devices provide a decrease in pressure output over 80 pulses that is less than 0.25 MPa.

The pressure output, as tested, was measured in-vitro using pressure sensors (hydrophones) positioned externally to the catheter balloon within the acoustic field generated by device pulse delivery. The device under test and hydrophones were immersed in de-gassed, deionized water maintained at approximately body temperature.

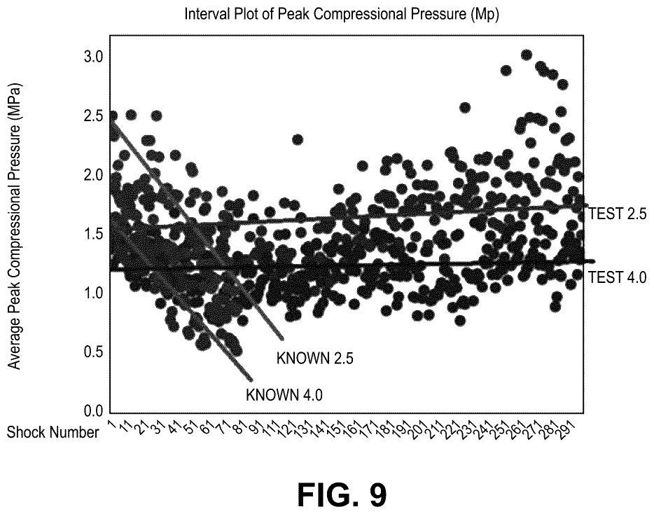

This concept is further demonstrated in FIG. 9, where the average pressure generated by 80 pulses of the KNOWN IVL devices (2.5 mm and 4.0 mm) with constant voltage magnitude is compared with the average pressure generated by the TEST IVL devices (2.5 mm and 4.0 mm) according to the voltage pulse and control method of FIG. 2.

As shown in FIG. 9, the KNOWN 2.5 mm and 4.0 mm devices both provide severely decreasing pressure output slope lines as the voltage pulses progress to 80 pulses. In contrast, the TEST 2.5 mm and 4.0 mm devices provide relatively flat, constant or stable pressure output slope lines as the voltage pulses progress to 300 pulses. Moreover, the slopes of the TEST pressure output lines appear to increase slightly as the voltage pulses progress which may be beneficial in cracking difficult calcified regions. Again, the KNOWN IVL devices have a pronounced and significant pressure output decay over 80 pulses, showing an approximate 60% decrease in pressure output. A significant pressure output decrease over 80 pulses may comprise more than a 10% decrease in pressure output as the therapeutic efficacy is reduced by a significant (more than 10%) amount. The TEST IVL devices do not have a pressure decay over 300 voltage pulses.

In summary, the IVL devices operated and controlled according to the present disclosure provide an increasing voltage to the voltage pulses until the upper voltage magnitude threshold is reached. Then, if the maximum number of voltage pulses has not been met, the voltage pulses may progress at the upper voltage magnitude threshold until 300, or more, voltage pulses (the predetermined maximum number of voltage pulses) have been executed, or the physical determines therapy is complete. This, as shown above, leads in certain embodiments, to constant and/or slightly increasing pressure output from each voltage pulse. The pressure output magnitudes, and associated slopes, may be manipulated in some embodiments by modifying the magnitude of each incremental increase in voltage. In some embodiments, the voltage magnitude may be incrementally increased as in FIG. 2. In others, the voltage magnitude may be increased incrementally for at least two series of voltage pulses, then held constant for one or more voltage pulses, then subsequent voltage pulse series may resume the incremental increase in magnitude. In other embodiments, the voltage magnitude may be decreased for one or more series of voltage pulses. All combinations of voltage increase, voltage decrease, and/or no change in voltage over a plurality of a series of voltage pulses in order to manipulate the resulting pressure output are within the scope of the present invention.

In addition, with reference to the above disclosure, various embodiments of the disclosure may provide a pressure output that is substantially the same for all balloon sizes, wherein the balloon sizes may be within a range from 2 mm to 4 mm outer diameter. In these embodiments, larger balloon sizes do not necessarily result in a lower pressure output than the pressure output of a relatively smaller balloon size.

The data of FIGS. 8 and 9 also demonstrate that IVL devices operated according to the present disclosure are also capable of stable operation and pressure output over a predetermined maximum number of voltage pulses comprising at least 300 voltage pulses. This is in contrast to the pronounced pressure decay of the KNOWN IVL devices over just 80 voltage pulses.

Moreover, the data of FIGS. 8 and 9 confirm that the IVL control system 22 shown in FIG. 1 is controllable such that the pressure output following an electrical arcing event between two spaced-apart electrodes can be controlled within upper and lower pressure magnitude thresholds or a pressure magnitude window. Further, the pressure output can be controlled using embodiments of the present disclosure in a pattern of increasing pressure over the procedure, decreasing pressure over the procedure, constant pressure over the procedure, and any combination thereof.

Examples of suitable processors may include one or more microprocessors, integrated circuits, system-on-a-chips (SoC), among others. Examples of suitable memory, may include one or more primary storage and/or non-primary storage (e.g., secondary, tertiary, etc. storage); permanent, semi-permanent, and/or temporary storage; and/or memory storage devices including but not limited to hard drives (e.g., magnetic, solid state), optical discs (e.g., CD-ROM, DVD-ROM), RAM (e.g., DRAM, SRAM, DRDRAM), ROM (e.g., PROM, EPROM, EEPROM, Flash EEPROM), volatile, and/or non-volatile memory; among others. Communication circuitry 58 includes components for facilitating processor operations, for example, suitable components may include transmitters, receivers, modulators, demodulators, filters, modems, analog/digital (AD or DA) converters, diodes, switches, operational amplifiers, and/or integrated circuits. In some embodiments, memory 26 may represent one or more memory devices operable for IVL therapy operation. For example, each memory (e.g., 154, 178) may be included as part of memory 26, shared, or isolated therefrom.

Within the present disclosure, consideration of a set of discharge electrodes has been discussed in the context of a pair of electrodes, one of which may serve as a cathode and the other of which may serve as an anode, at a given instance. However, the number of electrodes in a set may be greater than a pair, for example, including one or more cathodes communicating with one or more anodes. Additionally, devices, systems, and methods within the present disclosure may include more than one communicating group of electrodes, whether electrically arranged serially, parallel, or independently from each other.

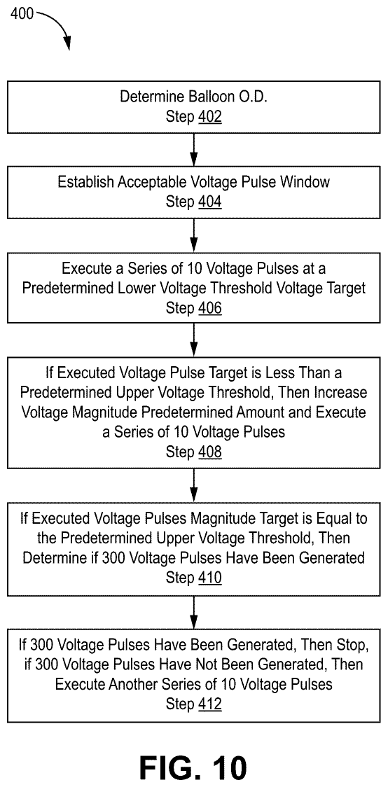

FIG. 10 provides an exemplary flowchart illustrating an exemplary method 400 of one embodiment of the present invention. Thus, step 402 provides for determination of the subject IVL device's balloon outer diameter or OD. This may be done with manual entry into the IVL control system discussed above. Alternatively, connecting the catheter with the IVL control system may provide automatic detection and determination of the balloon's OD. Step 404 provides for establishing an acceptable voltage pulse window, comprising as described above, predetermined lower and upper voltage magnitude thresholds which may be stored in the IVL control system. Step 406 provides for execution of a series of voltage pulses to be controlled and generated by the IVL control system, in an illustrative and exemplary case 10 pulses may be used, at the predetermined lower voltage magnitude threshold. Step 408 provides that if the IVL control system determines that the last executed series of voltage pulses was not executed at the predetermined upper voltage threshold target, then the IVL control system may instruct execution and generation of another series of voltage pulses. Step 410 provides that if the IVL control system determines that the last executed series of voltage pulses was executed at the predetermined upper voltage threshold target, then the IVL control system seeks to determine if the illustrative and exemplary predetermined maximum of 300 voltage pulses have been executed during the current therapy. If, according to step 412, 300 voltage pulses are determined to have been executed, the IVL control system stops the procedure, allowing no further voltage pulse generation. On the other hand, if 300 voltage pulses have not been executed, then the IVL control system instructs another series of voltage pulses to be executed and at the predetermined upper voltage threshold target magnitude.

In certain embodiments, the devices, systems and methods described herein may comprise a voltage pulsing frequency of 1 pulse/second, 2 pulses/second and/or 3 pulses/second. In some embodiments, the frequency or pulses/second generated by the described embodiments may be within the range of 1 to 5 pulses/second.

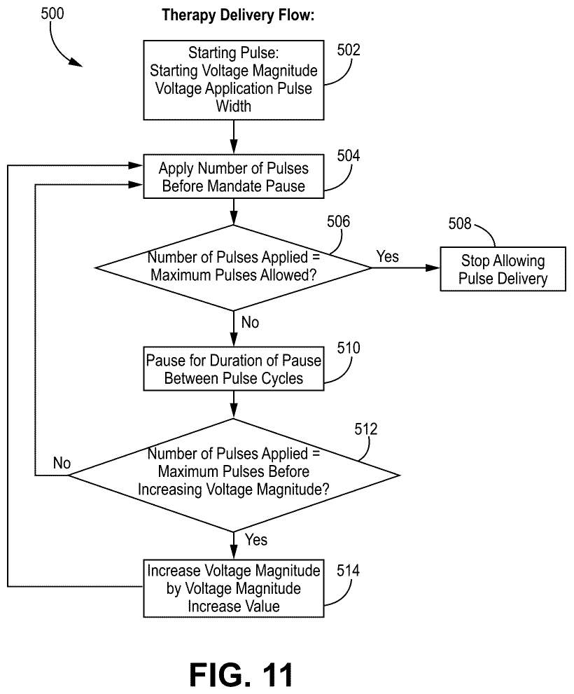

Turning now to FIG. 11, and with continued reference to FIGS. 1-10, some embodiments may comprise an IVL therapy flow 500 based on the control data comprising: the predetermined starting voltage; the upper voltage threshold; the width or duration of the generated and/or applied voltage pulse; the predetermined pause between adjacent voltage pulse series; the number of generated voltage pulses required before increasing voltage for subsequent voltage pulses; the magnitude of an increase in voltage of subsequent voltage pulses; the time gap between adjacent voltage pulses within a voltage pulse series; and the maximum number of voltage pulses. As discussed above, the control data may be stored in, and executed by, the control system 22. Alternatively, also as discussed above, the control data may be stored in a memory, processor, or an EPROM, wherein the control system 22 executes the therapy using the control data.

The executable IVL therapy flow 500 may be initiated in step 502 with generation of a starting voltage pulse and with the control data's predetermined duration or width of the voltage pulse. A predetermined number of voltage pulses (a series of voltage pulses) as provided by the control data may be executed in step 504, with a mandated or required pause of a predetermined duration before executing a subsequent or adjacent series of voltage pulses.

Next, in step 506, the number of generated voltage pulses is monitored or determined and compared with the control data's maximum number of voltage pulses allowed.

If, as in step 508, the maximum number of voltage pulses have been generated, then the control system 22 allows no further generation of voltage pulses. In some embodiments, additional voltage pulse generation may be allowed by swapping out the original catheter assembly 14 for a new catheter assembly, whereupon the number of voltage pulses generated restarts at zero.

If the maximum number of generated voltage pulses has not been reached, step 510 provides for the required pause between subsequent or adjacent series (or cycles) of the generated voltage pulses. The control data comprises the predetermined length or duration of the required pause between adjacent series of voltage pulses.

Following the mandated pause of step 510, step 512 determines if the total number of voltage pulses generated meets the control data's requirement for increasing the voltage magnitude for subsequent voltage pulses.

If the number of voltage pulses generated is insufficient to warrant an increase in voltage magnitude, then the operational flow returns to step 504 and subsequent steps as discussed above.

If, as in step 514, the number of voltage pulses generated is sufficient to warrant an increase in voltage magnitude, then the voltage magnitude is increased a predetermined amount for subsequent voltage pulses. From step 514, the operational flow returns to step 504 and subsequent steps as discussed above.

The description of devices, systems, and method and related applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

We claim:

1. An intravascular lithotripsy (“IVL”) catheter assembly comprising:
at least one set of electrodes for arrangement within a body lumen while disposed within an inflatable balloon or enclosure;
an electric pulse generation system for providing electrical energy to the at least one set of spaced-apart electrodes to generate spark for IVL therapy, the electric pulse generation system including an IVL control system comprising a processor for executing instructions based at least in part on a set of stored control data, and circuitry adapted for communication of signals based on operation of the processor, the IVL control system configured to:
generate an initial series of a predetermined number of voltage pulses to apply to the at least one set of spaced-apart electrodes, wherein the magnitude of each voltage pulse in the initial series of voltage pulses comprises a target voltage that is set at a predetermined lower voltage magnitude threshold,
after the generation of the initial series of voltage pulses, require a pause of a predetermined duration;
generate a subsequent series of a predetermined number of voltage pulses to apply to the at least one set of spaced-apart electrodes;
determine when the number of generated voltage pulses is sufficient to warrant an increase in the magnitude of subsequent voltage pulses and, upon such determination, increase the magnitude of the subsequent voltage pulses by a predetermined amount; and
generate another series of a predetermined number of voltage pulses to apply to the at least one set of spaced-apart electrodes,
wherein at least some of the generated voltage pulses generate an electrical arc between the spaced-apart electrodes of the at least one set of space-apart electrodes and wherein each electrical arc produces a pressure output.

2. The IVL catheter assembly of claim 1, wherein the predetermined lower voltage magnitude threshold is about 2850 volts.

3. The IVL catheter assembly of claim 1, wherein the IVL control system is further configured to determine when a predetermined upper threshold voltage magnitude has been reached.

4. The IVL catheter assembly of claim 3, wherein when the IVL control system is further configured to stop subsequent voltage magnitude increases when the predetermined upper threshold voltage magnitude is determined to have been reached.

5. The IVL catheter assembly of claim 3, wherein the predetermined upper threshold voltage magnitude is about 3250 volts.

6. The IVL catheter assembly of claim 1, wherein the duration of the pause is between about 5 seconds and about 20 seconds.

7. The IVL catheter assembly of claim 1, wherein the duration of the pause is about 10 seconds.

8. The IVL catheter assembly of claim 1, further comprising a predetermined duration of application of the voltage to the at least one set of electrodes.

9. The IVL catheter assembly of claim 8, wherein the duration or width of the application of the voltage is about 20 microseconds to about 30 microseconds.

10. The IVL catheter assembly of claim 9, wherein the duration or width of the application of the voltage is about 25 microseconds.

11. The IVL catheter assembly of claim 1, wherein the IVL control system is further configured to increase the magnitude of the subsequent voltage pulses by a predetermined amount of about 25 volts when the number of generated voltage pulses is determined by the IVL control system to be sufficient to warrant an increase in the magnitude of subsequent voltage pulses.

12. The IVL catheter assembly of claim 1, wherein if the IVL control system further comprises a predetermined maximum number of voltage pulses for the IVL catheter assembly that is within the range of 10 to 300 voltage pulses, and wherein the IVL control system is configured to prevent any further voltage pulses to be executed upon a determination that the maximum predetermined number of voltage pulses have been generated.

13. The IVL catheter assembly of claim 12, wherein the electrical arcs produce pressure outputs that do not decay or decrease on average more than 0.25 MPa across the predetermined maximum number of voltage pulses.

14. The IVL catheter assembly of claim 12, wherein the electrical arcs produce pressure outputs that do not decrease more than 10% across the predetermined maximum number of voltage pulses.

15. The IVL catheter assembly of claim 12, wherein the pressure output of a last voltage pulse of the predetermined maximum number of voltage pulses is greater than the pressure output of a first voltage pulse.

16. The IVL catheter assembly of claim 15, wherein the electrical arcs produce pressure outputs that comprise a slope of the pressure outputs of the generated voltage pulses over time, wherein the slope of the pressure outputs increases over time.

17. The IVL catheter assembly of claim 12, wherein the electrical arcs produce pressure outputs that comprise a slope of the pressure outputs of the generated voltage pulses over time, wherein a slope of the pressure output of the voltage pulses decreases over time.

18. The intravascular lithotripsy system of claim 12, wherein the electrical arcs produce pressure outputs that comprise a slope of the pressure output of the voltage pulses over time, wherein the slope of the pressure output of the voltage pulses indicates a substantially constant pressure magnitude output across the voltage pulses.

19. The intravascular lithotripsy system of claim 1, further comprising a plurality of the generated series of the predetermined number of voltage pulses.

20. The IVL catheter assembly of claim 19, wherein one or more of the series of generated voltage pulses in the plurality of generated series of voltage pulses comprises 10 voltage pulses.

21. The IVL catheter assembly of claim 20, wherein one or more of the series of generated voltage pulses in the plurality of generated series of voltage pulses comprises more than 10 voltage pulses.

22. The IVL catheter assembly of claim 19, wherein one or more of the series of generated voltage pulses in the plurality of generated series of voltage pulses comprises less than 10 voltage pulses.

23. The IVL catheter assembly of claim 1, wherein the stored control data comprises the predetermined lower voltage magnitude threshold, a predetermined duration of the application of the generated voltage pulse to the at least one set of electrodes, and the predetermined duration of the pause.

24. The IVL catheter assembly of claim 1, wherein the stored control data further comprises one or more of a maximum upper voltage threshold, a predetermined duration between adjacent voltage pulses within a series of voltage pulses, a predetermined number of voltage pulses required before an increase in voltage magnitude is warranted, and at least one predetermined magnitude to increase the voltage.

25. The IVL catheter assembly of claim 1, wherein the stored control data further comprises a maximum number of voltage pulses allowed for the IVL catheter assembly.

26. The IVL catheter assembly of claim 1, wherein the stored control data further comprises a current threshold value.

27. The IVL catheter assembly of claim 1, further comprising an EPROM configured to store the stored control data.

28. An intravascular lithotripsy (“IVL”) catheter assembly comprising:
at least one set of electrodes for arrangement within a body lumen while disposed within an inflatable balloon or enclosure;
an electric pulse generation system for providing electrical energy to the at least one set of electrodes to generate spark for IVL therapy, the electric pulse generation system including a current monitoring system and an IVL control system comprising a processor for executing instructions based at least in part on a set of stored control data, and circuitry adapted for communication of signals based on operation of the processor,
wherein the current monitoring system is configured to determine a current flow generated by the voltage pulse, and
wherein the IVL control system is configured to:
generate a voltage pulse to apply to the at least one set of electrodes responsive to an initial voltage magnitude setting comprising a predetermined magnitude of the voltage pulse and an initial duration setting comprising a predetermined duration of application of the voltage pulse to the spaced-apart electrodes,
compare the determined current flow with a predetermined current threshold; and
determine whether the initial voltage magnitude setting and the initial duration setting were sufficient to generate an electrical arc between the spaced-apart electrodes.

29. The IVL catheter assembly of claim 28, wherein the IVL control system is further configured to increase the initial voltage magnitude setting by a predetermined amount if the IVL control system determined that the initial voltage magnitude setting and the initial duration setting were not sufficient to generate an electrical arc.

30. The IVL catheter of claim 28, wherein the IVL control system is further configured to increase the initial duration setting by a predetermined amount if the IVL control system determined that the voltage magnitude setting and the initial duration setting were not sufficient to generate an electrical arc.

31. The IVL catheter of claim 28, wherein the IVL control system is further configured to increase the initial voltage magnitude setting by a predetermined amount, and increase the initial duration setting by a predetermined amount if the IVL control system determined that the initial voltage magnitude and duration settings were not sufficient to generate an electrical arc.

32. The IVL catheter assembly of claim 28, wherein the IVL control system is configured to generate a next voltage pulse to apply to the at least one set of electrodes with the initial voltage magnitude setting and the initial duration setting if the IVL control system determined that the initial voltage magnitude and initial duration settings generated an electrical arc, wherein the IVL control system is configured to determine whether the next voltage pulse did generate an electrical arc.

33. An intravascular lithotripsy (“IVL”) catheter assembly comprising:
at least one set of electrodes for arrangement within a body lumen while disposed within an inflatable balloon or enclosure;
an electric pulse generation system for providing electrical energy to the at least one set of electrodes to generate spark for IVL therapy, the electric pulse generation system including a current monitoring system and an IVL control system comprising a processor for executing instructions based at least in part on a set of stored control data, and circuitry adapted for communication of signals based on operation of the processor,
wherein the current monitoring system is configured to determine a current flow generated by each one of the voltage pulses in the series of voltage pulses, and wherein the IVL control system is configured to:
generate an initial series of voltage pulses to apply to the at least one set of electrodes responsive to an initial voltage magnitude setting comprising a predetermined magnitude of the voltage pulse and an initial duration setting comprising a predetermined duration of application of the initial series of voltage pulses to the spaced-apart electrodes,
calculate an average current flow from the determined current flows generated by each one of the voltage pulses in the initial series of voltage pulses,
compare the calculated average current flow with a predetermined current threshold, and
before initiation of a subsequent series of voltage pulses, determine whether the initial voltage magnitude setting and the initial duration setting were sufficient to generate electrical arcs for each voltage pulse in the initial series of voltage pulses.

34. The IVL catheter assembly of claim 33, wherein the IVL control system is further configured to increase the initial voltage magnitude setting by a predetermined amount if the IVL control system determines that the initial voltage magnitude setting and the initial duration setting were not sufficient to generate electrical arcs for each voltage pulse in the initial series of voltage pulses before initiation of a subsequent series of voltage pulses.

35. The IVL catheter of claim 33, wherein the IVL control system is further configured to increase the initial duration setting by a predetermined amount if the IVL control system determines that the initial voltage magnitude setting and the initial duration setting were not sufficient to generate electrical arcs for each voltage pulse in the initial series of voltage pulses before initiation of a subsequent series of voltage pulses.

36. The IVL catheter of claim 33, wherein the IVL control system is further configured to increase the initial voltage magnitude setting by a predetermined amount, and increase the initial duration setting by a predetermined amount if the IVL control system determines that the initial voltage magnitude and duration settings were not sufficient to generate electrical arcs for each voltage pulse in the initial series of voltage pulses before initiation of a subsequent series of voltage pulses.

37. The IVL catheter assembly of claim 33, wherein the IVL control system is configured to generate a subsequent series of voltage pulses to apply to the at least one set of electrodes if the IVL control system determined that the initial voltage magnitude and initial duration settings were sufficient to generate an electrical arc.

38. The IVL catheter of claim 37, wherein the IVL control system is further configured to:
calculate a subsequent average current flow from the determined current flow generated by each one of the voltage pulses in the subsequent series of voltage pulses,
compare the calculated subsequent average current flow with the predetermined current threshold, and
determine whether the initial voltage magnitude and duration settings were sufficient to generate electrical arcs for each voltage pulse in the subsequent series of voltage pulses.

39. The IVL catheter of claim 37, wherein the IVL control system is further configured to require a pause of a predetermined duration after the generated initial series of voltage pulses and before initiating the generation of the subsequent series of voltage pulses.

40. An intravascular lithotripsy (“IVL”) catheter assembly comprising:
at least one set of electrodes for arrangement within a body lumen while disposed within an inflatable balloon or enclosure;
an electric pulse generation system for providing electrical energy to the at least one set of spaced-apart electrodes to generate spark for IVL therapy, the electric pulse generation system including an IVL control system comprising a processor for executing instructions based at least in part on a set of stored control data, and circuitry adapted for communication of signals based on operation of the processor, the IVL control system configured to:
generate a predetermined number of voltage pulses to apply to the at least one set of spaced-apart electrodes,
wherein at least some of the generated voltage pulses generate an electrical arc between the spaced-apart electrodes of the at least one set of space-apart electrodes,
wherein each electrical arc produces a pressure output that does not decrease more than 10% across the generated voltage pulses.

41. A method for conducting intravascular lithotripsy (“IVL”) comprising:
providing a catheter assembly comprising:
at least one set of electrodes for arrangement within a body lumen while disposed within an inflatable balloon or enclosure;
an electric pulse generation system for providing electrical energy to the at least one set of electrodes to generate spark for IVL therapy, the electric pulse generation system including an IVL control system comprising a processor for executing instructions based at least in part on a set of stored control data, and circuitry adapted for communication of signals based on operation of the processor;
generating an initial series of a predetermined number of voltage pulses to apply to the at least one set of electrodes, wherein the magnitude of each voltage pulse in the initial series of voltage pulses comprises a target voltage that is set at a predetermined lower voltage magnitude threshold,
after the generation of the initial series of voltage pulses, requiring a pause of a predetermined duration;
generating a subsequent series of a predetermined number of voltage pulses to apply to the at least one set of electrodes;
determining when the number of generated voltage pulses is sufficient to warrant an increase in the magnitude of subsequent voltage pulses and, upon such determination, increase the magnitude of the subsequent voltage pulses by a predetermined amount; and
generating another series of a predetermined number of voltage pulses to the at least one set of electrodes.

42. A method for conducting intravascular lithotripsy (“IVL”) comprising:
providing a catheter assembly comprising:
at least one set of electrodes for arrangement within a body lumen while disposed within an inflatable balloon or enclosure;
an electric pulse generation system for providing electrical energy to the at least one set of electrodes to generate spark for IVL therapy, the electric pulse generation system including a current monitoring system and an IVL control system comprising a processor for executing instructions based at least in part on a set of stored control data, and circuitry adapted for communication of signals based on operation of the processor,
determining a current flow generated by each one of the voltage pulses in the series of voltage pulses;
generating an initial series of voltage pulses to apply to the at least one set of electrodes responsive to an initial voltage magnitude setting comprising a predetermined magnitude of the voltage pulse and an initial duration setting comprising a predetermined duration of application of the initial series of voltage pulses to the spaced-apart electrodes;
calculating an average current flow from the determined current flow generated by each one of the voltage pulses in the series of voltage pulses;
comparing the calculated average current flow with a predetermined current threshold; and
determining whether the initial voltage magnitude and duration settings were sufficient to generate electrical arcs for each voltage pulse in the initial series of voltage pulses before initiating a subsequent series of voltage pulses.

43. A method for conducting intravascular lithotripsy (“IVL”) comprising:
providing a catheter assembly comprising:
at least one set of electrodes for arrangement within a body lumen while disposed within an inflatable balloon or enclosure;
an electric pulse generation system for providing electrical energy to the at least one set of electrodes to generate spark for IVL therapy, the electric pulse generation system including a current monitoring system and an IVL control system comprising a processor for executing instructions based at least in part on a set of stored control data, and circuitry adapted for communication of signals based on operation of the processor;
determining a current flow generated by each one of the voltage pulses in the series of voltage pulses;
generating an initial series of voltage pulses to apply to the at least one set of electrodes responsive to an initial voltage magnitude setting comprising a predetermined magnitude of the voltage pulse and an initial duration setting comprising a predetermined duration of application of the initial series of voltage pulses to the spaced-apart electrodes;
calculating an average current flow from the determined current flow generated by each one of the voltage pulses in the series of voltage pulses;
comparing the calculated average current flow with a predetermined current threshold; and
determining whether the initial voltage magnitude and duration settings were sufficient to generate electrical arcs for each voltage pulse in the initial series of voltage pulses before initiating a subsequent series of voltage pulses.
